SEQUENCE LISTING (20 sequences)

SEQ ID NO: 1 - DNA, 530 bp, Artificial Sequence
  Synthetic
cagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacagccgcgtcagagcagctcaggttctgggagagggtagcgcagggtggccactgagaaccgggcaggtcacgcatcccccccttccctcccaccccctgccaagctctccctcccaggatcctctctggc

SEQ ID NO: 2 - DNA, 502 bp, Artificial Sequence
  Synthetic
tgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctc

SEQ ID NO: 3 - DNA, 5576 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccagatcatctagacagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacagccgcgtcagagcagctcaggttctgggagagggtagcgcagggtggccactgagaaccgggcaggtcacgcatcccccccttccctcccaccccctgccaagctctccctcccaggatcctctctggcttcgaaggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgagtttggacaaaccacaactagaatgcagtgaaaaaaatgctttatttgtgaaatttgtgatgctattgctttatttgtaaccattataagctgcaataaacaagttacgcgttgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctcatcgataggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcc

SEQ ID NO: 4 - DNA, 5235 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccagatcatctagacagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacagccgcgtcagagcagctcaggttctgggagagggtagcgcagggtggccactgagaaccgggcaggtcacgcatcccccccttccctcccaccccctgccaagctctccctcccaggatcctctctggcttcgaaatcgatcacgagactagcctcgagaagcttgatggccgccagtgtgatggatatctgcagaattcgcccttatggggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatccaccggtcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgagtttggacaaaccacaactagaatgcagtgaaaaaaatgctttatttgtgaaatttgtgatgctattgctttatttgtaaccattataagctgcaataaacaagttacgcgttgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctcatcgataggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcc

SEQ ID NO: 5 - DNA, 5928 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccagatcatctagacagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacagccgcgtcagagcagctcaggttctgggagagggtagcgcagggtggccactgagaaccgggcaggtcacgcatcccccccttccctcccaccccctgccaagctctccctcccaggatcctctctggcttcgaagctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaccggtcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgagtttggacaaaccacaactagaatgcagtgaaaaaaatgctttatttgtgaaatttgtgatgctattgctttatttgtaaccattataagctgcaataaacaagttacgcgttgctgtcctgaagtggacataggggcccgggttggaggaagaagactagctgagctctcggacccctggaagatgccatgacagggggctggaagagctagcacagactagagaggtaaggggggtaggggagctgcccaaatgaaaggagtgagaggtgacccgaatccacaggagaacggggtgtccaggcaaagaaagcaagaggatggagaggtggctaaagccagggagacggggtactttggggttgtccagaaaaacggtgatgatgcaggcctacaagaaggggaggcgggacgcaagggagacatccgtcggagaaggccatcctaagaaacgagagatggcacaggccccagaaggagaaggaaaagggaacccagcgagtgaagacggcatggggttgggtgagggaggagagatgcccggagaggacccagacacggggaggatccgctcagaggacatcacgtggtgcagcgccgagaaggaagtgctcatcgataggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcc

SEQ ID NO: 6 - DNA, 1300 bp, Homo sapiens
tttggctggtctaaagtgacctacactgccagtaggctggcatgtggctgccatttagctatggcaacaacagtgagtgggccacatgtccctcctcatccagaagactagcccaggcctattcacattaaagcagcaagttccacaagggaaagaagacttgtgtgagaccacttgaggcccaggcttaaaagtgacacacatgtcttcttctgtatgttattagccaaataaataagtcataaagcctgcccagattcaaggggtagggaaatagactccacttcttgagagggcctgcaaattcacattgcaaagaaatgtggatacaggaaggaaaataagttttatattcttgtaatcgatctatcgtgtataccctctatgtggtagtaactgtagatggtcatctgggaattaatccttattcacagtgtaaacttaattactcactaaaatatataaagcttttaatcatgtatgatattgagatttcatatcttggtacttaaaaatgtatcaaatgcttgctatgtgctcttgctataaagagctaattggtatgagggaaagccaggtatttactaatcaatgtagtgagtaaaatgacagaaaaattataagaagaacatgaatgagggcatttaatttaaactttaggaatcaagaaacgcttctcgaagcagtgattcctgccctgattcttaaataatgtgtaggcattagacaggaggataagtacaaaacgtggcatcatgagcaaaggcatggaaatggcccatgagcggagtgaacactggtttggggttgctccaaggtaaagttcaaaaagtatcctgcagtcaaccctttagcaccataaagaaactaaattatttagatgtttttatgagaacatatcaaaaagtacttttctgtcatccaatacttccacaaataaatcattagttcttgctaatcttcatctggcataaaaataatgacatcaactttcttcatgtaatttcccacttaattcctttactaggagcaatatcaattcctatatgacgtcattgccagcacctaccctgctcagaatggacaagtaaagaaaaacaaccatcaagaagataaaattgaatttgataatgaagtggacaaagtaaagcaggatgctaattgtgttaatccacttggtgccccagaaaagctccctgaagcaaaggaacaggctgaaggttctgaacccacgagtggcactgaggggccagaacattctgtcaatggtcctgcaagtccagctttaaatcaaggttca

SEQ ID NO: 7 - DNA, 1200 bp, Homo sapiens
taggaaaagacataaatgaggaaactccaaacctcctgttagctgttatttctatttttgtagaagtaggaagtgaaaataggtatacagtggattaattaaatgcagcgaaccaatatttgtagaagggttatattttactactgtggaaaaatatttaagatagttttgccagaacagtttgtacagacgtatgcttattttaaaattttatctcttattcagtaaaaaacaacttctttgtaatcgttatgtgtgtatatgtatgtgtgtatgggtgtgtgtttgtgtgagagacagagaaagagagagaattctttcaagtgaatctaaaagcttttgcttttcctttgtttttatgaagaaaaaatacattttatattagaagtgttaacttagcttgaaggatctgtttttaaaaatcataaactgtgtgcagactcaataaaatcatgtacatttctgaaatgacctcaagatgtcctccttgttctactcatatatatctatcttatatagtttactattttacttctagagatagtacataaaggtggtatgtgtgtgtatgctactacaaaaaagttgttaactaaattaacattgggaaatcttatattccatatattagcatttagtccaatgtctttttaagcttatttaattaaaaaatttccagtgagcttatcatgctgtctttacatggggttttcaattttgcatgctcgattattccctgtacaatatttaaaatttattgcttgatacttttgacaacaaattaggttttgtacaattgaacttaaataaatgtcattaaaataaataaatgcaatatgtattaatattcattgtataaaaatagaagaatacaaacatatttgttaaatatttacatatgaaatttaatatagctatttttatggaatttttcattgatatgaaaaatatgatattgcatatgcatagttcccatgttaaatcccattcataactttcattaaagcatttactttgaatttctccaatgcttagaatgtttttaccaggaatggatgtcgctaatcataataaaattcaaccattatttttttcttgtttataatacattgtgttatatgttcaaatatgaaatgtgtatgcacctattgaaatatgtttaatgcatttattaacatttgcaggacacttttacaggcc

SEQ ID NO: 8 - DNA, 6275 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccagaaccggttcatctagatttggctggtctaaagtgacctacactgccagtaggctggcatgtggctgccatttagctatggcaacaacagtgagtgggccacatgtccctcctcatccagaagactagcccaggcctattcacattaaagcagcaagttccacaagggaaagaagacttgtgtgagaccacttgaggcccaggcttaaaagtgacacacatgtcttcttctgtatgttattagccaaataaataagtcataaagcctgcccagattcaaggggtagggaaatagactccacttcttgagagggcctgcaaattcacattgcaaagaaatgtggatacaggaaggaaaataagttttatattcttgtaatcgatctatcgtgtataccctctatgtggtagtaactgtagatggtcatctgggaattaatccttattcacagtgtaaacttaattactcactaaaatatataaagcttttaatcatgtatgatattgagatttcatatcttggtacttaaaaatgtatcaaatgcttgctatgtgctcttgctataaagagctaattggtatgagggaaagccaggtatttactaatcaatgtagtgagtaaaatgacagaaaaattataagaagaacatgaatgagggcatttaatttaaactttaggaatcaagaaacgcttctcgaagcagtgattcctgccctgattcttaaataatgtgtaggcattagacaggaggataagtacaaaacgtggcatcatgagcaaaggcatggaaatggcccatgagcggagtgaacactggtttggggttgctccaaggtaaagttcaaaaagtatcctgcagtcaaccctttagcaccataaagaaactaaattatttagatgtttttatgagaacatatcaaaaagtacttttctgtcatccaatacttccacaaataaatcattagttcttgctaatcttcatctggcataaaaataatgacatcaactttcttcatgtaatttcccacttaattcctttactaggagcaatatcaattcctatatgacgtcattgccagcacctaccctgctcagaatggacaagtaaagaaaaacaaccatcaagaagataaaattgaatttgataatgaagtggacaaagtaaagcaggatgctaattgtgttaatccacttggtgccccagaaaagctccctgaagcaaaggaacaggctgaaggttctgaacccacgagtggcactgaggggccagaacattctgtcaatggtcctgcaagtccagctttaaatcaaggttcagaaggtcgtggatcactacttacgtgcggtgatgtagaagagaatccgggtccggtcgacatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgagtttggacaaaccacaactagaatgcagtgaaaaaaatgctttatttgtgaaatttgtgatgctattgctttatttgtaaccattataagctgcaataaacaagttacgcgttaggaaaagacataaatgaggaaactccaaacctcctgttagctgttatttctatttttgtagaagtaggaagtgaaaataggtatacagtggattaattaaatgcagcgaaccaatatttgtagaagggttatattttactactgtggaaaaatatttaagatagttttgccagaacagtttgtacagacgtatgcttattttaaaattttatctcttattcagtaaaaaacaacttctttgtaatcgttatgtgtgtatatgtatgtgtgtatgggtgtgtgtttgtgtgagagacagagaaagagagagaattctttcaagtgaatctaaaagcttttgcttttcctttgtttttatgaagaaaaaatacattttatattagaagtgttaacttagcttgaaggatctgtttttaaaaatcataaactgtgtgcagactcaataaaatcatgtacatttctgaaatgacctcaagatgtcctccttgttctactcatatatatctatcttatatagtttactattttacttctagagatagtacataaaggtggtatgtgtgtgtatgctactacaaaaaagttgttaactaaattaacattgggaaatcttatattccatatattagcatttagtccaatgtctttttaagcttatttaattaaaaaatttccagtgagcttatcatgctgtctttacatggggttttcaattttgcatgctcgattattccctgtacaatatttaaaatttattgcttgatacttttgacaacaaattaggttttgtacaattgaacttaaataaatgtcattaaaataaataaatgcaatatgtattaatattcattgtataaaaatagaagaatacaaacatatttgttaaatatttacatatgaaatttaatatagctatttttatggaatttttcattgatatgaaaaatatgatattgcatatgcatagttcccatgttaaatcccattcataactttcattaaagcatttactttgaatttctccaatgcttagaatgtttttaccaggaatggatgtcgctaatcataataaaattcaaccattatttttttcttgtttataatacattgtgttatatgttcaaatatgaaatgtgtatgcacctattgaaatatgtttaatgcatttattaacatttgcaggacacttttacaggccatcgatgcagcggccgctgcaggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcc

SEQ ID NO: 9 - DNA, 540 bp, Homo sapiens
caaaggcatggaaatggcccatgagcggagtgaacactggtttggggttgctccaaggtaaagttcaaaaagtatcctgcagtcaaccctttagcaccataaagaaactaaattatttagatgtttttatgagaacatatcaaaaagtacttttctgtcatccaatacttccacaaataaatcattagttcttgctaatcttcatctggcataaaaataatgacatcaactttcttcatgtaatttcccacttaattcctttactaggagcaatatcaattcctatatgacgtcattgccagcacctaccctgctcagaatggacaagtaaagaaaaacaaccatcaagaagataaaattgaatttgataatgaagtggacaaagtaaagcaggatgctaattgtgttaatccacttggtgccccagaaaagctccctgaagcaaaggaacaggctgaaggttctgaacccacgagtggcactgaggggccagaacattctgtcaatggtcctgcaagtccagctttaaatcaaggttca

SEQ ID NO: 10 - DNA, 453 bp, Homo sapiens
taggaaaagacataaatgaggaaactccaaacctcctgttagctgttatttctatttttgtagaagtaggaagtgaaaataggtatacagtggattaattaaatgcagcgaaccaatatttgtagaagggttatattttactactgtggaaaaatatttaagatagttttgccagaacagtttgtacagacgtatgcttattttaaaattttatctcttattcagtaaaaaacaacttctttgtaatcgttatgtgtgtatatgtatgtgtgtatgggtgtgtgtttgtgtgagagacagagaaagagagagaattctttcaagtgaatctaaaagcttttgcttttcctttgtttttatgaagaaaaaatacattttatattagaagtgttaacttagcttgaaggatctgtttttaaaaatcataaactgtgtgcagactcaataaaatca

SEQ ID NO: 11 - DNA, 4754 bp, Artificial Sequence
  Synthetic
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccagatcatctagacaaaggcatggaaatggcccatgagcggagtgaacactggtttggggttgctccaaggtaaagttcaaaaagtatcctgcagtcaaccctttagcaccataaagaaactaaattatttagatgtttttatgagaacatatcaaaaagtacttttctgtcatccaatacttccacaaataaatcattagttcttgctaatcttcatctggcataaaaataatgacatcaactttcttcatgtaatttcccacttaattcctttactaggagcaatatcaattcctatatgacgtcattgccagcacctaccctgctcagaatggacaagtaaagaaaaacaaccatcaagaagataaaattgaatttgataatgaagtggacaaagtaaagcaggatgctaattgtgttaatccacttggtgccccagaaaagctccctgaagcaaaggaacaggctgaaggttctgaacccacgagtggcactgaggggccagaacattctgtcaatggtcctgcaagtccagctttaaatcaaggttcagaaggtcgtggatcactacttacgtgcggtgatgtagaagagaatccgggtccggtcgacatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatgagtttggacaaaccacaactagaatgcagtgaaaaaaatgctttatttgtgaaatttgtgatgctattgctttatttgtaaccattataagctgcaataaacaagttacgcgttaggaaaagacataaatgaggaaactccaaacctcctgttagctgttatttctatttttgtagaagtaggaagtgaaaataggtatacagtggattaattaaatgcagcgaaccaatatttgtagaagggttatattttactactgtggaaaaatatttaagatagttttgccagaacagtttgtacagacgtatgcttattttaaaattttatctcttattcagtaaaaaacaacttctttgtaatcgttatgtgtgtatatgtatgtgtgtatgggtgtgtgtttgtgtgagagacagagaaagagagagaattctttcaagtgaatctaaaagcttttgcttttcctttgtttttatgaagaaaaaatacattttatattagaagtgttaacttagcttgaaggatctgtttttaaaaatcataaactgtgtgcagactcaataaaatcaatcgatgcatgcaggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcc

SEQ ID NO: 12 - DNA, 54 bp, Artificial Sequence
  Synthetic
gaaggtcgtggatcactacttacgtgcggtgatgtagaagagaatccgggtccg

SEQ ID NO: 13 - DNA, 268 bp, Artificial Sequence
  Synthetic
cacttcaggacagcatgtttgctgcctccagggatcctgtgtccccgagctgggaccaccttatattcccagggccggttaatgtggctctggttctgggtacttttatctgtcccctccaccccacagtggggccactagggacaggattggtgacagaaaagccccatccttaggcctcctccttcctagtctcctgatattgggtctaacccccacctcctgttaggcagattccttatctggtgacacacccccatttcctgga

SEQ ID NO: 14 - DNA, 23 bp, Artificial Sequence
  Synthetic
ggggccactagggacaggattgg

SEQ ID NO: 15 - DNA, 20 bp, Homo sapiens
gcaagtccagctttaaatca

SEQ ID NO: 16 - DNA, 20 bp, Homo sapiens
ataacagctaacaggaggtt

SEQ ID NO: 17 - DNA, 20 bp, Homo sapiens
cataggaaaagacataaatg

SEQ ID NO: 18 - DNA, 20 bp, Homo sapiens
agctttaaatcaaggttcat

SEQ ID NO: 19 - DNA, 20 bp, Homo sapiens
tatgaaccttgatttaaagc

SEQ ID NO: 20 - DNA, 20 bp, Homo sapiens
tagaaataacagctaacagg

(19) United States
(12) Patent Application Publication
(10) Pub. No.: US 2024/0216534 A1
(43) Pub. Date: Jul. 4, 2024

(54) DIRECT RAAV-MEDIATED IN VIVO GENE EDITING OF HEMATOPOIETIC STEM CELLS

(71) Applicant: University of Massachusetts, Boston, MA (US)

(72) Inventors: Terence Flotte, Worcester, MA (US); Allison Keeler-Klunk, Worcester, MA (US)

(73) Assignee: University of Massachusetts, Boston, MA (US)

(21) Appl. No.: 18/288,227

(22) PCT Filed: Apr. 26, 2022
(86) PCT No.: PCT/US2022/026307

§ 371 Date: Oct. 25, 2023

Related U.S. Application Data
(60) Provisional application No. 63/179,748, filed on Apr. 26, 2021.

(51) Int. Cl. A61K 48/00; A61K 38/42; A61P 7/00; C07K 14/005; C12N 9/20; C12N 15/11; C12N 15/86

(52) U.S. Cl. CPC A61K 48/005; A61K 38/42; A61P 7/00; C07K 14/005; C12N 9/20; C12N 15/11; C12N 15/86; C12N 2310/20; C12N 2750/14122; C12N 2750/14143

(57) ABSTRACT

Aspects of the disclosure relate to compositions and methods for gene editing in a cell or subject. In some aspect, the present disclosure provides an isolated nucleic acid comprising an expression construct comprising transgene encoding a gene product flanked by a 5′ homology arm and a 3′ homology arm, wherein the expression construct is flanked by adeno-associated virus (AAV) inverted terminal repeats (ITRs).

### RELATED APPLICATIONS

This application is a national stage filing under 35 U.S.C. § 371 of international PCT application PCT/US2022/026307, filed Apr. 26, 2022, which claims priority under 35 U.S.C. § 119(e) to U.S. provisional patent application, U.S. Ser. No. 63/179,748, filed Apr. 26, 2021, the entire contents of each of which are incorporated by reference herein.

### REFERENCE TO AN ELECTRONIC SEQUENCE LISTING

The contents of the electronic sequence listing (U012070166US01-SEQ-KZM.txt; Size: 45,425 bytes; and Date of Creation: Oct. 23, 2023) is herein incorporated by reference in its entirety.

### BACKGROUND

Gene editing of hematopoietic stem cells (HCSs) has progressed to clinical stage and represents a tremendously promising platform for future gene therapy for hemoglobinopathies such as sickle cell disease (Hgb SS disease). However, there are inherent practical limitations to scaling up such approaches to make them accessible to global populations most affected by these disorders.

### SUMMARY

Aspects of the disclosure relate to compositions and methods for gene editing in a cell or subject. In some embodiments, the gene editing occurs in vitro. In some embodiments, the gene editing occurs in vivo. The disclosure is based, in part, on isolated nucleic acids (e.g., expression constructs) and rAAVs engineered to 1) express one or more gene products that are flanked by homology arms specific for a genomic safe harbor (GSH) locus or a genomic locus for a gene, and 2) target stem cell populations of a subject. In some embodiments, compositions described herein are directly targeted (e.g., administered directly to) a target tissue or population of cells (e.g., hematopoietic stem cells, pluripotent stem cells, etc.) of a subject, for example by direct injection into the target tissue or population of cells (e.g., into bone marrow). In some embodiments, compositions described herein are useful for in vivo or ex vivo homology directed repair (HDR) of certain genes associated with disease, for example genes associated with hemoglobinopathies.

Accordingly, in some aspects, the disclosure provides an isolated nucleic acid comprising an expression construct comprising transgene encoding a gene product flanked by a 5′ homology arm and a 3′ homology arm, wherein the expression construct is flanked by adeno-associated virus (AAV) inverted terminal repeats (ITRs).

In some embodiments, a gene product comprises a protein or inhibitory nucleic acid. In some embodiments, a gene product comprises a therapeutic protein or a reporter protein. In some embodiments, a therapeutic protein is useful for treating a hemoglobinopathy. In some embodiments, the hemoglobinopathy is sickle cell disease. In some embodiments, the therapeutic protein is a Hemoglobin Subunit Beta.

In some embodiments, homology arms are specific for a human genomic locus. In some embodiments, a human genomic locus comprises a genomic safe harbor (GSH) site. In some embodiments, a GSH site is an AAVIS GSH site. In some embodiments, the 5′ AAVS1 homology arm comprises a nucleic acid sequence of SEQ ID NO: 1. In some embodiments, the 3′ AAVS1 homology arm comprises a nucleic acid sequence of SEQ ID NO: 2.

In some embodiments, an expression cassette further comprises a promoter operably linked to the transgene. In some embodiments, a promoter comprises a CMV promoter, EF1a promoter, or a myeloproliferative sarcoma virus enhancer, negative control region deleted, d1587rev primer-binding site substituted (MND) promoter.

In some aspects, the present disclosure provides an isolated nucleic acid comprising an expression construct comprising transgene encoding a gene product flanked by a 5′ homology arm and a 3′ homology arm, wherein the expression construct is flanked by adeno-associated virus (AAV) inverted terminal repeats (ITRs). In some embodiments, the isolated nucleic acid further comprises a nucleic acid sequence encoding a 2A peptide, wherein the nucleic acid sequence encoding the 2A peptide is located between the 5′ homology arm and the transgene. In some embodiments, the isolated nucleic acid further comprises a stop codon located at the 5′ end of the 3′ homology arm. In some embodiments, the 5′ and 3′ homology arms are specific for a genomic locus of a gene. In some embodiments, the 5′ and 3′ homology arms are specific for a genomic locus of CD45. In some embodiments, the CD45 is human CD45. In some embodiments, the 5′ homology arm specific for CD45 comprises the nucleic acid sequence as set forth in SEQ ID NO: 6 or SEQ ID NO: 9. In some embodiments, the 3′ homology arm specific for CD45 comprises the nucleic acid sequence as set forth in SEQ ID NO: 7 or SEQ ID NO: 10.

In some embodiments, AAV ITRs are AAV2 ITRs. In some embodiments, at least one of the AAV ITRs comprises a mutant ITR, such as a deltaITR (AITR).

In some embodiments, the isolated nucleic acid comprises any one of SEQ ID NOs: 3-5, 8 or 11.

In some aspects, the disclosure provides a recombinant adeno-associated virus (rAAV) comprising an isolated nucleic acid as described herein; and an AAV capsid protein.

In some embodiments, an AAV capsid protein is of a serotype selected from AAV1. AAV2. AAV3, AAV4, AAV5, AAV6, AAV7, AAV8, AAV9, or a variant thereof. In some embodiments, an AAV capsid protein targets bone or bone marrow cells. In some embodiments, an AAV capsid protein is an AAV6 capsid protein.

In some aspects, the disclosure provides a pharmaceutical composition comprising an isolated nucleic acid or the rAAV as described herein. In some embodiments, the pharmaceutical composition further comprises one or more (e.g., 1, 2, 3, 4, 5, or more) guideRNAs (gRNAs).

In some embodiments, one or more gRNAs comprise a region of complementarity with the homology arms of the isolated nucleic acid or rAAV (or a region of complementarity with a GSH locus, for example AAVIS locus). In some embodiments, one or more gRNAs specifically bind to a genomic safe harbor (GSH) locus. In some embodiments, a GSH locus comprises an AAVIS locus. In some embodiments, the gRNAs specifically bind to the target sequence of the AAVIS locus as set forth in SEQ ID NO: 14. In some embodiments, the gRNAs specifically bind to the target sequence of human CD45 locus as set forth in any one of SEQ ID NOs: 15-20.

In some embodiments, a pharmaceutical composition further comprises an RNA-guided nuclease (RGN) or an isolated nucleic acid encoding an RGN. In some embodiments, an RGN comprises a Cas9 protein or variant thereof. In some embodiments, the RGN is a SpCas9.

In some embodiments, the disclosure provides a method for in vivo homology directed repair (HDR), the method comprising administering an isolated nucleic acid, rAAV, or pharmaceutical composition as described herein, to a subject.

In some embodiments, the disclosure provides a method for in vitro homology directed repair (HDR), the method comprising administering an isolated nucleic acid, rAAV, or pharmaceutical composition as described herein to an ex vivo cell. In some embodiments, the method comprises introducing the ex vivo cell into a subject.

In some embodiments, a subject is a mammal. In some embodiments, a subject is a human. In some embodiments, a subject is characterized as having, or being at risk of having, a hemoglobinopathy. In some embodiments, the hemoglobinopathy is sickle cell disease.

In some embodiments, a cell is a mammalian cell. In some embodiments, a cell is a human cell. In some embodiments, a cell is a hematopoietic stem cell (HSC).

In some embodiments, the method further comprising administering to the subject or the cell a gRNA targeting the genomic safe harbor (GSH) locus and an RNA-guided nuclease (RGN). In some embodiments, the gRNA and the RGN are administered to the subject or the cell concurrently with the isolated nucleic acid, or the rAAV as described herein. In some embodiments, the gRNA and the RGN are administered to the subject or the cell subsequently to the administration of the isolated nucleic acid or the rAAV as described herein. In some embodiments, the gRNA and the RGN are administered to the subject or the cell prior to administration of the isolated nucleic acid or the rAAV as described herein.

In some embodiments, the method further comprising administering to the subject or the cell a gRNA targeting the genomic safe harbor (GSH) locus and a nucleic acid encoding a RNA-guided nuclease (RGN). In some embodiments, the gRNA and the nucleic acid encoding RGN are administered to the subject or the cell concurrently with the isolated nucleic acid or the rAAV as described herein. In some embodiments, the gRNA and the nucleic acid encoding RGN are administered to the subject or the cell subsequently to the administration of isolated nucleic acid or the rAAV as described herein. In some embodiments, the gRNA and the RGN are administered to the subject or the cell prior to administration of the isolated nucleic acid of or the rAAV as described herein.

### BRIEF DESCRIPTION OF THE DRAWINGS

FIGS. 1A-1D show ex vivo editing of hematopoietic stem cell (HSC) using rAAVs engineered to express GFP that are flanked by homology arms specific for a AAVS1 genomic safe harbor (GSH) locus. FIG. 1A shows the experimental design of editing HSCs. FIG. 1B are graphs showing flow cytometry of GFP positive cells after gene editing by AAV6-AAVS1-MND-GFP, AAV6-AAVS1-CMV-GFP, or AAV6-AAVS1-E1fa-GFP. FIG. 1C shows quantification of GFP positive cells from the flow cytometry graphs in FIG. 1B. FIG. 1D shows ddPCR validation of targeted transgene integration at the AAVS1 GSH locus after gene editing by AAV6-AAVS1-MND-GFP, AAV6-AAVS1-CMV-GFP, or AAV6-AAVS1-E1fa-GFP.

FIGS. 2A-2D show in vivo differentiation of human HSCs after gene editing using AAV6-AAVS1-MND-GFP in immunodeficient mice. FIG. 2A shows the experimental design of in vivo differentiation of human HSCs in NBSGW mouse strain after gene editing. FIG. 2B shows ddPCR evaluation of edited cells in bone marrow and spleen. FIG. 2C shows flow cytometry evaluation of GFP+/hCD15+ cells in the bone marrow and the spleen after differentiation. FIG. 2D shows lineage distribution in the mice engrafted with unedited and edited cells in different tissues. Edited cells belong to myeloid lineage.

FIGS. 3A-3E how in vivo HSC editing using AAV6-AAVS1-MND-GFP. FIG. 3A shows the experimental design of in vivo HSC editing using AAV6-AAVS1-MND-GFP. FIG. 3B shows intraosseous injection in B6 mice resulted in systemic biodistribution of AAV6 24 hours post injection as tested by ddPCR. FIG. 3C shows that higher viral genomes (VGs) in the engrafted cells was observed in the injected BM as compared to the contralateral BM. FIG. 3D shows that, in addition to intraosseous injection in vivo, systemic injection can be an alternative approach to administer AAV6-AAVS1-MND-GFP. FIG. 3E shows the mechanisms of how systemic administration of AAV6-AAVS1-MND-GFP can lead to gene editing in HSCs.

FIGS. 4A-4G show gene editing of T cells using CD45 promoter hijacking approach coupled with SpCas9 mediated editing. FIG. 4A shows the AAV construct having CD45 homology arms flanking a nucleic acid encoding a 2A peptide, a transgene, and a stop codon and gene editing using this construct results in incorporation of the transgene downstream of CD45 gene such that the native CD45 promoter can drive the expression of CD45 and the transgene. FIG. 4B shows experimental design of T cell gene editing using this construct. FIG. 4C-4D shows various guide RNA targeting CD45 were tested and the gRNA3 and gRNA4 Showed high CD45 knockout score. gRNA4 was selected for subsequent testing in in vivo editing of T cells. FIG. 4E shows the experimental design of editing T cells using SpCas9, gRNA targeting CD45, and AAV vector having the Homology-directed repair (HDR) donor sequences. FIGS. 4F-4G shows the PCR results after gene editing. gRNA4 showed successful HDR.

### DETAILED DESCRIPTION

Aspects of the disclosure relate to compositions and methods for gene editing in a cell or subject. In some embodiments, the gene editing occurs in vitro. In some embodiments, the gene editing occurs ex vivo. In some embodiments, the gene editing occurs in vivo. The disclosure is based, in part, on isolated nucleic acids (e.g., expression constructs) and rAAVs engineered to 1) express one or more gene products that are flanked by homology arms specific for a genomic safe harbor (GSH) locus or a genomic locus for a gene, and 2) target a population of cells (e.g., hematopoietic stem cells, lymphocytes, etc.) of a subject. In some embodiments, compositions described herein are directly targeted (e.g., administered directly to) a target tissue or population of cells (e.g., hematopoietic stem cells, pluripotent stem cells, etc.) of a subject, for example by direct injection into the target tissue or population of cells (e.g., into bone marrow). In some embodiments, compositions described herein are administered systemically to a subject. In some embodiments, compositions described herein are useful for in vivo or ex vivo homology directed repair (HDR) of certain genes associated with disease, for example genes associated with hemoglobinopathies.

### Isolated Nucleic Acids

The disclosure relates, in some aspects, to an isolated nucleic acid comprising two adeno-associated virus (AAV) inverted terminal repeats (ITRs) flanking an expression cassette, wherein the expression cassette comprises a transgene encoding a gene product. An expression cassette, as used herein, refers to component of vector DNA comprising a protein coding sequence to be expressed by a cell having the vector and its regulatory sequences. Once delivered to the target cell, the expression cassette directs the cell's machinery to make RNA and/or protein(s).

A “nucleic acid” sequence refers to a DNA or RNA sequence. In some embodiments, proteins and nucleic acids of the disclosure are isolated. As used herein, the term “isolated” means artificially produced. As used herein with respect to nucleic acids, the term “isolated” means: (i) amplified in vitro by, for example, the polymerase chain reaction (PCR); (ii) recombinantly produced by cloning; (iii) purified, for example, by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

In some embodiments, the present disclosure also provides an isolated nucleic acid comprising an expression construct comprising transgene encoding a gene product flanked by a 5′ homology arm and a 3′ homology arm, wherein the expression construct is flanked by adeno-associated virus (AAV) inverted terminal repeats (ITRs). The isolated nucleic acid of the present disclosure is designed to facilitate gene editing (e.g., insertion of a transgene to a genomic locus) by homologous recombination.

Homologous recombination refers to a type of genetic recombination in which genetic information is exchanged between two similar or identical molecules of double-stranded or single-stranded nucleic acids (e.g., DNA or RNA). It is used by cells to repair breaks that occur on both strands of DNA, known as double-strand breaks (DSB), in a process called homologous recombinational repair (HRR). Homologous recombination has been previous described to perform gene editing (e.g., insertion) at a genomic locus. In some embodiments, the homologous recombination described in the present disclosure is a RNA-guided nuclease (RGN mediated homologous recombination. Non-limiting examples of RGN include Cas9 and a variant thereof (e.g., SpCas9, SaCas9, Cas9 Nickases, High-Fidelity Cas9, eSpCas9, HypaCas9, FokI-Fused dCas9, xCas9 and SpRY/SpG, etc), or Cas12a. In some embodiments, the RNA-guided nuclease is Cas9 or a variant thereof. In some embodiments, the Cas9 is SpCas9. RGN mediated gene editing has been previously described, see, e.g., Souza et al., RNA-guided gene editing, Nature Methods volume 10, page 189 (2013). RGD-mediated (e.g., Cas9-mediated) homologous recombination describes a method to make an desired change to the genome. The method includes making a DNA double-strand break using Cas9 at a genomic locus. A homologous repair template containing the genome modification of interest and the homology arms. The double-strand break is repaired by homologous recombination with the modified template supplied guided by a guide RNA targeting the genomic locus. Accordingly, genetic modification such as insertions, deletions, point mutants, in-frame GFP fusions, or any combination thereof can be achieved.

A guide RNA (gRNA), as used herein, is a RNA molecule that functions as guides for DNA or RNA-targeting nucleases (RGN), which they form complexes with, which results in deletion, insertion or otherwise alteration of the targeted RNA or DNA. The gRNA can occur naturally or can be chemically synthesized. gRNAs serve important functions, but can also be designed to be used for targeted editing, such as with CRISPR-Cas9 and CRISPR-Cas12. In some embodiments, the gRNA is at least 80, at least 85, at least 90, at least 95, at least 100, at least 105, at least 110, at least 115, at least 120, at least 125, at least 130, at least 135, at least 140, at least 150 or more base pairs in length. In some embodiments, the gRAN comprises a region of complementarity to the target RNA that is 7, 8, 9, 10, 11, 12, 13, 14, 15, 16, 17, 18, 19, 20, 21, 22, 23, 24, 25, 26, 27, 28, 29, 30, 35, 40, 45, 50, or more base pairs in length. In some embodiments, the gRAN comprises a region of complementarity to the target RNA that is 8 to 30 base pairs in length, 10 to 15 base pairs in length, 10 to 20 base pairs in length, 15 to 25 base pairs in length, 19 to 21 base pairs in length, or 21 to 23 base pairs in length.

In some embodiments, gRNA comprises a region of complementarity to a target region in a genomic locus (e.g., AAVS1 locus or CD45 locus). In some embodiments, the region of complementarity is at least 80%, at least 85%, at least 90%, at least 91%, at least 92%, at least 93%, at least 94%, at least 95%, at least 96%, at least 97%, at least 98%, at least 99%, or 100% complementary to a target region in the genomic locus (e e.g., AAVS1 locus or CD45 locus). In some embodiments, a complementary nucleotide sequence need not be 100% complementary to that of its target to be specifically hybridizable or specific for the genomic locus (e.g., AAVS1 locus or CD45 locus).

In some embodiments, a gRNA comprises a region of complementarity to (AAVS1 locus or CD45 locus) sequence and the region of complementarity is in the range of 8 to 15, 8 to 30, 8 to 40, or 10 to 50, or 5 to 50, or 5 to 40 nucleotides in length. In some embodiments, the region of complementarity is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary to at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a genomic locus (e.g., AAVS1 locus or CD45 locus). In some embodiments, the region of complementarity comprises a nucleotide sequence that contains no more than 1, 2, 3, 4, or 5 base mismatches compared to the complementary portion of the genomic locus (e.g., AAVS1 locus or CD45 locus). In some embodiments, the region of complementarity comprises a nucleotide sequence that has up to 3 mismatches over 15 bases, up to 2 mismatches over 10 bases, or up to 1 mismatch over 5 bases. In some embodiments, the present disclosure provides an isolated nucleic acid comprising an expression construct comprising transgene encoding a gene product flanked by a 5′ homology arm and a 3′ homology arm. Homology arms, as used herein, refers to two nucleic acid sequences that are homologous to a genomic locus of interest, which are called 5′ homology arm and 3′ homology arm. After delivery of the isolated nucleic acid to the target cell when a double strand break is introduced to the genomic locus of interest, the homology arms recombines with the genome sequence by homologous recombination, thereby introducing the transgene into the genomic locus of interest (e.g., Kan et al., (2014) The mechanism of gene targeting in human somatic cells. PLOS Genet 10: e1004251). In some embodiments, a 5′ homology arm and/or a 3′ homology arm is between 300 and 2000 bp, between 400 and 1800 bp, between 500 and 1600 bp, between 600 and 1500 bp, between 700 and 1400 bp, between 800 and 1200 bp, between 400 and 1000 bp, between 500 and 900 bp, between 300 and 800 bp, between 300 and 700 bp, between 300 and 600 bp, between 300 and 500 bp, between 400 and 500 bp, between 450 and 550 bp, between 500 and 600 bp, between 600 and 700 bp, between 700 and 800 bp, between 800 and 900 bp, between 500 and 1000 bp, between 500 and 1500 bp, between 1000 and 1500 bp, between 1100 and 1300 bp, or between 1000 and 1300 bp.

In some embodiments, the genomic locus where the transgene is introduced is a genomic safe harbor (GSH) locus. A genomic safe harbor (GSH) locus, as used herein, refer to a locus in the genome able to accommodate the integration of new genetic material in a manner that ensures that the newly inserted genetic elements: (i) function predictably and (ii) do not cause alterations of the host genome posing a risk to the host cell or organism. GSHs are thus ideal sites for transgene insertion (see, e.g., Papapetrou et al., Gene Insertion Into Genomic Safe Harbors for Human Gene Therapy, Mol Ther, 2016 April; 24(4):678-84; Pavani et al., Targeted Gene Delivery: Where to Land, Front. Genome Ed., 20 Jan. 2021). Non-limiting GSH locus include AAVS1, CCR5, and Rosa26. In some embodiments, the genomic safe harbor locus is an AAVS1 site. The AAVS1 locus (chromosome 19 q13.42) was historically identified as the preferential integration site of wild-type AAV in human cell lines (Kotin et al., Characterization of a preferred site on human chromosome 19q for integration of adeno-associated virus DNA by non-homologous recombination. EMBO J. 1992, 11, 5071-5078). It encodes the PPP1R12C gene. Stable and corrective editing of patients' HSC at this locus has been obtained by integrating a transgene cassette with (Diez et al., Therapeutic gene editing in CD34(+) hematopoietic progenitors from Fanconi anemia patients. EMBO Mol. Med. 9, 1574-1588. (2017)) or without an exogenous promoter (De Ravin et al., Targeted gene addition in human CD34(+) hematopoietic cells for correction of X-linked chronic granulomatous disease. Nat. Biotechnol. 34, 424-429. (2016)). In some embodiments, the AAVS1 locus comprises a nucleic acid sequence at least 80%, at least 85%, at least 90%, at least 91%, at least 92%, at least 93%, at least 94%, at least 95%, at least 96%, at least 97%, at least 98%, at least 99%, or 100% identical to the nucleic acid as set forth in SEQ ID NO: 13. In some embodiments, the isolated nucleic acid comprises a 5′ homology arm and a 3′ homology arm specific for a human genomic locus (e.g., a genomic safe harbor (GSH) site). In some embodiments, the isolated nucleic acid comprises a 5′ homology arm and a 3′ homology arm specific AAVS1 GSH site. In some embodiments, a 5′ homology arm and/or a 3′ homology arm specific for AAVS1 GSH site is between 300 and 2000 bp, between 400 and 1800 bp, between 500 and 1600 bp, between 600 and 1500 bp, between 700 and 1400 bp, between 800 and 1200 bp, between 400 and 1000 bp, between 500 and 900 bp, between 300 and 800 bp, between 300 and 700 bp, between 300 and 600 bp, between 300 and 500 bp, between 400 and 500 bp, between 450 and 550 bp, between 500 and 600 bp, between 600 and 700 bp, between 700 and 800 bp, between 800 and 900 bp, between 500 and 1000 bp, between 500 and 1500 bp, between 1000 and 1500 bp, between 1100 and 1300 bp, or between 1000 and 1300 bp. In some embodiments, the 5′ AAVS1 homology arm comprises a nucleic acid sequence at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleic acid sequence as set forth in SEQ ID NO: 1. In some embodiments, the 3′ AAVS1 homology arm comprises a nucleic acid sequence at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleic acid sequence as set forth as set forth in SEQ ID NO: 2.

In some embodiments, a transgene encoding a gene product is operably linked to a promoter. A “promoter” refers to a DNA sequence recognized by the synthetic machinery of the cell, or introduced synthetic machinery, required to initiate the specific transcription of a gene. The phrases “operatively linked,” “operatively positioned,” “under control” or “under transcriptional control” means that the promoter is in the correct location and orientation in relation to the nucleic acid to control RNA polymerase initiation and expression of the gene. As used herein, a transgene (e.g., coding sequence) and regulatory sequences are said to be “operably linked” when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly, two or more coding regions are operably linked when they are linked in such a way that their transcription from a common promoter results in the expression of two or more proteins having been translated in frame.

Generally, a promoter can be a constitutive promoter, inducible promoter, or a tissue-specific promoter.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the chimeric cytomegalovirus chimeric cytomegalovirus (CMV)/Chicken β-actin (CB) promoter (CBA promotor), the SV40 promoter, the dihydrofolate reductase promoter, the β-actin promoter, the phosphoglycerol kinase (PGK) promoter, and the EF1a promoter [Invitrogen]. In some embodiments, a promoter is an RNA pol II promoter. In some embodiments, a promoter is the chimeric cytomegalovirus chimeric cytomegalovirus (CMV)/Chicken β-actin (CB) promoter (CBA promoter). In some embodiments, a promoter is an RNA pol III promoter, such as U6 or H1. In some embodiments, the constitutive promoter is a CMV promoter. In some embodiments, a promoter is a chicken beta-actin (CB) promoter. A chicken beta-actin promoter may be a short chicken beta-actin promoter or a long chicken beta-actin promoter. In some embodiments, a promoter (e.g., a chicken beta-actin promoter) comprises an enhancer sequence, for example a cytomegalovirus (CMV) enhancer sequence. A CMV enhancer sequence may be a short CMV enhancer sequence or a long CMV enhancer sequence. In some embodiments, a promoter comprises a long CMV enhancer sequence and a long chicken beta-actin promoter. In some embodiments, a promoter comprises a short CMV enhancer sequence and a short chicken beta-actin promoter. However, the skilled artisan recognizes that a short CMV enhancer may be used with a long CB promoter, and a long CMV enhancer may be used with a short CB promoter (and vice versa). In some embodiments, the isolated nucleic acid comprises 5′ homology arm and 3′ homology arm specific to GSH locus comprises a CMV promoter. In some embodiments, the isolated nucleic acid comprises 5′ homology arm and 3′ homology arm specific to GSH locus comprises an EF1a promoter. In some embodiments, the isolated nucleic acid comprises 5′ homology arm and 3′ homology arm specific to GSH locus comprises MND promoter (see, e.g., Sather et al., Development of B-lineage Predominant Lentiviral Vectors for Use in Genetic Therapies for B Cell Disorders, Mol Ther. 2011 March; 19(3): 515-525).

Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: retinoschisin proximal promoter, interphotoreceptor retinoid-binding protein enhancer (RS/IRBPa), rhodopsin kinase (RK), liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (α-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

An isolated nucleic acid described herein may also contain one or more introns. In some embodiments, at least one intron is located between the promoter/enhancer sequence and the transgene. In some embodiments, an intron is a synthetic or artificial (e.g., heterologous) intron. Examples of synthetic introns include an intron sequence derived from SV-40 (referred to as the SV-40 T intron sequence) and intron sequences derived from chicken beta-actin gene. In some embodiments, a transgene described by the disclosure comprises one or more (1, 2, 3, 4, 5, or more) artificial introns. In some embodiments, the one or more artificial introns are positioned between a promoter and a transgene.

In some embodiments, the genomic locus where the transgene is introduced is a genomic locus of a gene. In some embodiments, the genomic locus where the transgene is introduced is the genomic locus of CD45. In some embodiments, the CD45 is human CD45. In some embodiments, the isolated nucleic acid comprises a 5′ homology arm and a 3′ homology arm specific for human CD45. In some embodiments, a 5′ homology arm and/or a 3′ homology arm specific for human CD45 is between 300 and 2000 bp, between 400 and 1800 bp, between 500 and 1600 bp, between 600 and 1500 bp, between 700 and 1400 bp, between 800 and 1200 bp, between 400 and 1000 bp, between 500 and 900 bp, between 300 and 800 bp, between 300 and 700 bp, between 300 and 600 bp, between 300 and 500 bp, between 400 and 500 bp, between 450 and 550 bp, between 500 and 600 bp, between 600 and 700 bp, between 700 and 800 bp, between 800 and 900 bp, between 500 and 1000 bp, between 500 and 1500 bp, between 1000 and 1500 bp, between 1100 and 1300 bp, or between 1000 and 1300 bp. In some embodiments, the 5′ CD45 homology arm comprises a nucleic acid sequence at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleic acid sequence as set forth in SEQ ID NOs: 6 or 9. In some embodiments, the 3′ CD45 homology arm comprises a nucleic acid sequence at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to the nucleic acid sequence as set forth as set forth in SEQ ID NOs: 7 or 10. In some embodiments, the 5′ CD45 homology arm comprises a nucleic acid sequence as set forth in SEQ ID NO: 6 or 9. In some embodiments, the 3′ CD45 homology arm comprises a nucleic acid sequence as set forth in SEQ ID NO: 7 or 10.

In some embodiments, the isolated nucleic acid further comprises a 2A peptide coding sequence located between the 5′ homology arm (e.g., CD45 5′ homology arm) and the transgene. A 2A self-cleaving peptides, or 2A peptides, is a class of 18-22 aa-long peptides, which can induce ribosomal skipping during translation of a protein in a cell. Non-limiting examples of 2A peptide include T2A peptide, P2A peptide, E2A peptide, or F2A peptide. In some embodiments, the 2A peptide is a T2A peptide. In some embodiments, the T2A peptide coding sequence comprises the nucleic acid sequence as set forth in SEQ ID NO: 12. In some embodiments, the 3′ homology arm (e.g., CD45 3′ homology arm) starts with a stop codon coding sequence. In some embodiments, the stop codon is UAG, UAA or UGA.

In some embodiments, the isolated nucleic acid comprises an expression construct comprising transgene encoding a gene product flanked by a 5′ homology arm and a 3′ homology arm specific for a genomic locus of a gene (e.g., CD45) does not comprise a promoter. In some embodiments, an isolated nucleic acid comprising an expression construct comprising transgene encoding a gene product flanked by a 5′ homology arm and a 3′ homology arm specific for a genomic locus of a gene (e.g., CD45), once integrates into the genomic locus (e.g., CD45 locus), hijacks the endogenous promoter of the gene (e.g., endogenous CD45 promoter) such that the promoter of the gene is driving the transcription of the endogenous gene and the transgene to generate a multicistronic mRNA.

In some embodiments, the isolated nucleic acid described herein comprises an expression construct comprising transgene encoding a gene product flanked by a 5′ homology arm and a 3′ homology arm. One of ordinary skill in the art would be able to choose a gene product based on the purpose of interest. In some embodiments, the gene product comprises a protein or inhibitory nucleic acid. In some embodiments, the gene product is a therapeutic protein or a reporter protein. In some embodiments, suitable reporter proteins include but are not limited to eGFP, eYFP, cCFP, mKate2, mCherry, mPlum, mGrape2, mRaspberry, mGrape1, mStrawberry, mTangerine, mBanana, mHoneydew, tdTomato. beta-galactosidase (encoded by LacZ), horseradish peroxidase, or luciferase. Reporter proteins may be used for imaging and/or diagnostic purposes. In some embodiments, the gene product is GFP. In some embodiments the isolated nucleic acid described herein comprises an expression construct comprising a transgene, and the transgene does not encode a reporter protein (e.g., GFP). Instead, the transgene encodes a therapeutic protein or a inhibitory nucleic acid.

In some embodiments, the gene product is a therapeutic protein. In some embodiments, the therapeutic protein is useful for treating a hemoglobinopathy. A hemoglobinopathy, as used herein, refers to a group of genetic disorders in which there is abnormal production or structure of the hemoglobin molecule. Non-limiting examples of hemoglobinopathy includes hemoglobin C disease, hemoglobin S-C disease, sickle cell anemia, and thalassemias. In some embodiments, the hemoglobinopathy is sickle cell anemia. In some embodiments, the gene product is Hemoglobin Subunit Beta (HBB).

In some embodiments, the gene product of the isolated nucleic acid described herein encodes an inhibitory nucleic acid. Inhibitory nucleic acids and there use in silencing gene expression are familiar to those skilled in the art and are described elsewhere herein. In some embodiments, the RNAi molecule targets an endothelia-function related gene described elsewhere herein. In some embodiments, an inhibitory nucleic acid include a microRNA, siRNA, or shRNA.

An isolated nucleic acid described by the disclosure may encode a transgene that further comprises a polyadenylation (poly A) sequence. In some embodiments, a transgene comprises a poly A sequence is a rabbit beta-globin (RBG) poly A sequence.

In some embodiments, the isolated nucleic acid comprises inverted terminal repeats flanking the expression construct. The isolated nucleic acids of the disclosure may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs).

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid further comprises a region (e.g., a second region, a third region, a fourth region, etc.) comprising a second AAV ITR. In some embodiments, an isolated nucleic acid encoding a transgene is flanked by AAV ITRs (e.g., in the orientation 5′-ITR-transgene-ITR-3′). In some embodiments, the AAV ITRs are selected from the group consisting of AAV1 ITR, AAV2 ITR, AAV3 ITR, AAV4 ITR, AAV5 ITR, and AAV6 ITR. In some embodiments, the second ITR is a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ΔTRS ITR, or AITR). Without wishing to be bound by any particular theory, a rAAV vector comprising an ITR lacking a functional TRS produces a self-complementary rAAV vector, for example as described by McCarthy (2008) Molecular Therapy 16(10): 1648-1656. In some embodiments, vectors described herein comprise one or more AAV ITRs, and at least one ITR is an ITR variant of a known AAV serotype ITR. In some embodiments, the AAV ITR variant is a synthetic AAV ITR (e.g., AAV ITRs that do not occur naturally). In some embodiments, the AAV ITR variant is a hybrid ITR (e.g., a hybrid ITR comprises sequences derived from ITRs of two or more different AAV serotypes).

In some embodiments, an isolated nucleic acid (e.g., a rAAV vector) as described herein comprises, from 5′ to 3′ order: a 5′ AAV ITR, an AAVS1 5′ homology arm, a CMV promoter, a transgene of interest, an AAVS1 3′ homology arm, and a 3′ AAV ITR.

In some embodiments, an isolated nucleic acid (e.g., a rAAV vector) as described herein comprises, from 5′ to 3′ order: a 5′ AAV ITR, an AAVS1 5′ homology arm, a EF1a promoter, a transgene of interest, an AAVS1 3′ homology arm, and a 3′ AAV ITR.

In some embodiments, an isolated nucleic acid (e.g., a rAAV vector) as described herein comprises, from 5′ to 3′ order: a 5′ AAV ITR, a AAVS1 5′ homology arm, a MND promoter, a transgene of interest, an AAVS1 3′ homology arm, and a 3′ AAV ITR.

In some embodiments, an isolated nucleic acid (e.g., a rAAV vector) as described herein comprises, from 5′ to 3′ order: a 5′ AAV ITR, a CD45 5′ homology arm, a T2A peptide coding sequence, a transgene of interest, a stop codon, a CD45 3′ homology arm, and a 3′ AAV ITR.

In some embodiments, an isolated nucleic acid (e.g., an AAV vector) comprises a nucleic acid sequence at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to any one of the nucleic acid sequence as set forth in SEQ ID NOs: 3-5, 8 or 11. In some embodiments, an isolated nucleic acid (e.g., an AAV vector) does not encode a reporter protein (e.g., GFP), but encodes a therapeutic protein or an inhibitory nucleic acid of interest.

Recombinant Adeno-Associated Viruses (rAAVs)

In some aspects, the disclosure provides isolated adeno-associated viruses (AAVs). As used herein with respect to AAVs, the term “isolated” refers to an AAV that has been artificially produced or obtained. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s) (e.g., blood lineage cells). The AAV capsid is an important element in determining these tissue-specific targeting capabilities (e.g., tissue tropism). Thus, an rAAV having a capsid appropriate for the tissue being targeted can be selected.

In some embodiments, the rAAV of the present disclosure comprises a capsid protein containing the isolated nucleic acid described herein.

Methods for obtaining recombinant AAVs having a desired capsid protein are well known in the art. (See, for example, US 2003/0138772, the contents of which are incorporated herein by reference in their entirety). Typically the methods involve culturing a host cell which contains a nucleic acid sequence encoding an AAV capsid protein; a functional rep gene; a recombinant AAV vector composed of AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein has a tropism for blood lineage cells. In some embodiments, an AAV capsid protein targets blood lineage cells (e.g., hematopoietic stem cells. T cells, etc.).

In some embodiments, an AAV capsid protein is of an AAV serotype selected from the group consisting of AAV1, AAV2, AAV3, AAV4, AAV5, AAV6, AAV7, AAV8, AAV9, AAV9.hr. AAVrh8, AAVrh10, AAVrh39, AAVrh43, AAV.PHP, and variants of any of the foregoing. In some embodiments, the AAV capsid protein is of an AAV6 serotype.

In some embodiments, the rAAV described herein is a single stranded AAV (ssAAV). An ssAAV, as used herein, refers to an rAAV with the coding sequence and complementary sequence of the transgene expression cassette on separate strands and are packaged in separate viral capsids.

The components to be cultured in the host cell to package an rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the disclosure relates to a host cell containing a nucleic acid that comprises a coding sequence encoding a transgene. A “host cell” refers to any cell that harbors, or is capable of harboring, a substance of interest. Often a host cell is a mammalian cell. In some embodiments, a host cell is a photoreceptor cell, retinal pigment epithelial cell, keratinocyte, corneal cell, and/or a tumor cell. A host cell may be used as a recipient of an AAV helper construct, an AAV vector, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation. In some embodiments, the host cell is a mammalian cell, a yeast cell, a bacterial cell, an insect cell, a plant cell, or a fungal cell. In some embodiments, the host cell is a hematopoietic stem cell, a lymphocyte (e.g., T cell or B cell), or loid cells (e.g., macrophages, NK cells).

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an AAV vector (comprising a transgene flanked by ITR elements) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). Non-limiting examples of vectors suitable for use with the disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing. AAV DNA replication, synthesis of cap expression products, and AAV capsid assembly. Viral-based accessory functions can be derived from any of the known helper viruses such as adenovirus, herpes virus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. In some embodiments, a vector is a viral vector, such as an rAAV vector, a lentiviral vector, an adenoviral vector, a retroviral vector, an anellovirus vector (e.g., Anellovirus vector as described in US20200188456A1), etc. Thus, the term includes cloning and expression vehicles, as well as viral vectors. In some embodiments, useful vectors are contemplated to be those vectors in which the nucleic acid segment to be transcribed is positioned under the transcriptional control of a promoter.

The present disclosure also provides pharmaceutical compositions comprising the isolated nucleic acid or the rAAV described herein. In some embodiments, the pharmaceutical composition further comprises one or more guide RNAs (gRNAs). In some embodiments, the one or more gRNAs comprise a region of complementarity the genomic locus that the homology arms are specific for. In some embodiments, the one or more gRNAs specifically bind to a genomic safe harbor (GSH) locus. In some embodiments, the GSH locus comprises an AAVIS locus. In some embodiments, the gRNAs specifically bind to the target sequence of the AAVIS locus as set forth in SEQ ID NO: 13 or 14. In some embodiments, the one or more gRNAs specifically bind to CD45. In some embodiments, the CD45 is human CD45. In some embodiments, the gRNAs specifically bind to the target sequence of human CD45 locus as set forth in any one of SEQ ID NOs: 15-20.

In some embodiments, a gRNA comprises a region of complementarity to a region in a genomic locus (e.g., AAVS1 locus or CD45 locus) sequence as set forth in any one of SEQ ID NOs: 13-20. In some embodiments, the region of complementarity in a gRNA is at least 80%, at least 85%, at least 90%, at least 91%, at least 92%, at least 93%, at least 94%, at least 95%, at least 96%, at least 97%, at least 98%, at least 99%, or 100% complementary to a region in the genomic locus (e e.g., AAVS1 locus or CD45 locus) sequence as set forth in any one of SEQ ID NOs: 13-20. In some embodiments, a complementary nucleotide sequence need not be 100% complementary to that of its target to be specifically hybridizable or specific for the genomic locus (e.g., AAVS1 locus or CD45 locus) sequence as set forth in any one of SEQ ID NOs: 13-20.

In some embodiments, the region of complementarity is complementary to at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a genomic locus (e.g., AAVS1 locus or CD45 locus) target sequences as set forth in any one of SEQ ID NOs: 13-20. In some embodiments, the region of complementarity comprises a nucleotide sequence that contains no more than 1, 2, 3, 4, or 5 base mismatches compared to the complementary portion of the genomic locus (e.g., AAVS1 locus or CD45 locus) target sequences as set forth in any one of SEQ ID NOs: 13-20. In some embodiments, the region of complementarity comprises a nucleotide sequence that has up to 3 mismatches over 15 bases, up to 2 mismatches over 10 bases, or up to 1 mismatch over 5 bases to the target sequences as set forth in any one of SEQ ID NOs: 13-20.

In some embodiments, a gRNA comprise a nucleotide sequence that is complementary (e.g., at least 85%, at least 90%, at least 95%, or 100%) to a target RNA sequence as set forth in SEQ ID NOs: 13-20. In some embodiments, a gRNA comprises a nucleotide sequence that is at least 85%, at least 90%, at least 95%, or 100% complementary to the sequence as set forth in SEQ ID NOs: 13-20. In some embodiments, a gRNA comprise at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides that is complementary to the sequence as set forth in SEQ ID NOs: 13-20.

In some embodiments, the pharmaceutical composition further comprising: (i) an RNA-guided nuclease (RGN); or (ii) an isolated nucleic acid encoding an RGN. Non-limiting examples of RGN include Cas9 and a variant thereof (e.g., SpCas9, SaCas9, Cas9 Nickases, High-Fidelity Cas9, eSpCas9, HypaCas9, FokI-Fused dCas9, xCas9 and SpRY/SpG, etc), or Cas12a. In some embodiments, the RNA-guided nuclease is Cas9 or a variant thereof. In some embodiments, the Cas9 is SpCas9. RGNs and their corresponding coding sequences are known in the art and can be selected by one of ordinary skill in the art.

### AAV-Mediated Gene Editing

The isolated nucleic acids, vectors, rAAVs, and compositions comprising the isolated nucleic acid described herein, the vectors described herein, or the rAAV described herein of the disclosure may be delivered to a subject in compositions according to any appropriate methods known in the art. For example, an rAAV, preferably suspended in a physiologically compatible carrier (e.g., in a composition), may be administered to a subject, i.e. host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human. In some embodiments, the subject is a human. In some embodiments, the subject is a non-human mammal. In some embodiments, the non-human mammal include but a not limited to mouse, rat, pig, cow, sheep, goat, donkey, camel, llama, monkey, etc.

In some aspects, the present disclosure provides a method for in vivo homology directed repair (HDR), the method comprising administering the isolated nucleic acid, the rAAV, or the pharmaceutical composition described herein, to a subject. In some embodiments, the subject is a mammal. In some embodiments, the subject is a human. In some embodiments, the subject is

In some aspects, the present disclosure provides a method for in vitro homology directed repair (HDR), the method comprising administering the isolated nucleic acid, the rAAV, or the pharmaceutical composition described herein, to an ex vivo cell; and, optionally, introducing the cell into a subject.

In some embodiments, the present disclosure provides a method for treating a disease in a subject. The disease can be any of the diseases that require gene replacement therapy, or inhibitor treatment (e.g., administration of inhibitory nucleic acid). As used herein, treating or treatment refer to achieving a therapeutic benefit in a subject, e.g., to extend the lifespan of a subject, to improve and/or reverse in the subject one or more symptoms of disease, or to slow disease progression.

In some embodiments, the method further comprising administering to the subject or the cell a gRNA targeting the genomic safe harbor (GSH) locus (e.g., AAVIS) and an RNA-guided nuclease (RGN) (e.g., SpCas9) or the RNA-guided nuclease (RGN) coding sequence (e.g., SpCas9 coding sequence). In some embodiments, a gRNA targeting the genomic safe harbor (GSH) locus (e.g., AAVIS) and an RNA-guided nuclease (RGN) (e.g., SpCas9) or the RNA-guided nuclease (RGN) coding sequence (e.g., SpCas9 coding sequence) are administered to the cell or subject concurrently with the isolated nucleic acid, rAAV, or pharmaceutical composition described herein. In some embodiments, a gRNA targeting the genomic safe harbor (GSH) locus (e.g., AAVIS) and an RNA-guided nuclease (RGN) (e.g., SpCas9) or the RNA-guided nuclease (RGN) coding sequence (e.g., SpCas9 coding sequence) are administered to the cell or subject sequentially to the administration of the isolated nucleic acid, rAAV, or pharmaceutical composition described herein. In some embodiments, a gRNA targeting the genomic safe harbor (GSH) locus (e.g., AAVIS) and an RNA-guided nuclease (RGN) (e.g., SpCas9) or the RNA-guided nuclease (RGN) coding sequence (e.g., SpCas9 coding sequence) are administered to the cell or subject prior to the administration of the isolated nucleic acid, rAAV, or pharmaceutical composition described herein.

In some embodiments, the method further comprising administering to the subject or the cell a gRNA targeting the genomic locus of a gene (e.g., human CD45) and an RNA-guided nuclease (RGN) (e.g., SpCas9) or the RNA-guided nuclease (RGN) coding sequence (e.g., SpCas9 coding sequence). In some embodiments, a gRNA targeting the genomic locus of a gene (e.g., human CD45) and an RNA-guided nuclease (RGN) (e.g., SpCas9) or the RNA-guided nuclease (RGN) coding sequence (e.g., SpCas9 coding sequence) are administered to the cell or subject concurrently with the isolated nucleic acid, rAAV, or pharmaceutical composition described herein. In some embodiments, a gRNA targeting the genomic locus of a gene (e.g., human CD45) and an RNA-guided nuclease (RGN) (e.g., SpCas9) or the RNA-guided nuclease (RGN) coding sequence (e.g., SpCas9 coding sequence) are administered to the cell or subject sequentially to the administration of the isolated nucleic acid, rAAV, or pharmaceutical composition described herein. In some embodiments, a gRNA targeting the genomic locus of a gene (e.g., human CD45) and an RNA-guided nuclease (RGN) (e.g., SpCas9) or the RNA-guided nuclease (RGN) coding sequence (e.g., SpCas9 coding sequence) are administered to the cell or subject prior to the administration of the isolated nucleic acid, rAAV, or pharmaceutical composition described herein.

In some embodiments, administration of an isolated nucleic and/or an rAAV as described herein result in homologous recombination of the genomic locus to integrate the transgene into the genome of the cell or the subject. Delivery of the rAAVs to a mammalian subject may be by, for example, direct injection to the BM (e.g., intraosseous injection).

Alternatively, delivery of the rAAVs to a mammalian subject may be by intramuscular injection or by administration into the bloodstream of the mammalian subject. Administration into the bloodstream may be by injection into a vein, an artery, or any other vascular conduit. Non-limiting exemplary methods of intramuscular administration of the rAAV include Intramuscular (IM) Injection and Intravascular Limb Infusion. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. In some embodiments, an rAAV or a composition (e.g., composition containing the isolated nucleic acid or the rAAV) as described in the disclosure is administered by intravitreal injection. In some embodiments, an rAAV or a composition (e.g., composition containing the isolated nucleic acid or the rAAV) as described in the disclosure is administered by intraocular injection. In some embodiments, an rAAV or a composition (e.g., composition containing the isolated nucleic acid or the rAAV) as described in the disclosure is administered by subretinal injection. In some embodiments, an rAAV or a composition (e.g., composition containing the isolated nucleic acid or the rAAV) as described in the disclosure is administered by intravenous injection. In some embodiments, an rAAV or a composition (e.g., composition containing the isolated nucleic acid or the rAAV) as described in the disclosure is administered by intramuscular injection. In some embodiments, an rAAV or a composition (e.g., composition containing the isolated nucleic acid or the rAAV) as described in the disclosure is administered by intratumoral injection.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

In some embodiments, a composition further comprises a pharmaceutically acceptable carrier. Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, parachlorophenol, and poloxamers (non-ionic surfactants) such as Pluronic® F-68. Suitable chemical stabilizers include gelatin and albumin.

The rAAVs or the compositions (e.g., composition containing the isolated nucleic acid or the rAAV described herein) are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraosseous to the bone marrow), intravenous, intramuscular, subcutaneous, intradermal, intratumoral, and other parental routes of administration. Routes of administration may be combined, if desired.

The dose of rAAV virions required to achieve a particular “gene editing effect,” e.g., the units of dose in genome copies/per kilogram of body weight (GC/kg), will vary based on several factors including, but not limited to: the route of rAAV virion administration, the level of gene or RNA expression required to achieve a gene editing effect, the specific disease or disorder being treated, and the stability of the gene or RNA product. One of skill in the art can readily determine an rAAV virion dose range to treat a patient having a particular disease or disorder based on the aforementioned factors, as well as other factors that are well known in the art.

An effective amount of rAAVs or composition (e.g., composition containing the isolated nucleic acid or the rAAV described herein) is an amount sufficient to target infect an animal, target a desired tissue (e.g., bone marrow, etc.). In some embodiments, an effective amount of an rAAV is administered to the subject during a pre-symptomatic stage of a disease. In some embodiments, a subject is administered an rAAV or composition after exhibiting one or more signs or symptoms of a disease. In some embodiments, the effective amount will depend primarily on factors such as the species, age, weight, health of the subject, and the tissue to be targeted, and may thus vary among animal and tissue. For example, an effective amount of the rAAV is generally in the range from about 1 ml to about 100 ml of solution containing from about 10⁶ to 10¹⁶ genome copies (e.g., from 1×10⁶ to 1×10¹⁶, inclusive). In some embodiments, an effective amount of an rAAV ranges between 1×10⁹ and 1×10¹⁴ genome copies of the rAAV. In some cases, a dosage between about 10¹¹ to 10¹² rAAV genome copies is appropriate.

In some embodiments, rAAV compositions are formulated to reduce aggregation of AAV particles in the composition, particularly where high rAAV concentrations are present (e.g., ˜10¹³ GC/mL or more). Methods for reducing aggregation of rAAVs are well-known in the art and, include, for example, addition of surfactants, pH adjustment, salt concentration adjustment, etc. (Sec, e.g., Wright F R, et al., Molecular Therapy (2005) 12, 171-178, the contents of which are incorporated herein by reference.)

Formulation of pharmaceutically-acceptable excipients and carrier solutions are well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf-life, as well as other pharmacological considerations will be contemplated by one skilled in the art of preparing such pharmaceutical formulations, and as such, a variety of dosages and treatment regimens may be desirable.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that it is easily syringed. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes are generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

### EXAMPLE

### Example 1: Gene Editing of HSPCs Under AAVS1 Safe Harbor Locus

This example describes in situ gene modification that enables a direct targeting of the Hematopoietic stem/progenitor cells (HSPCs) ex vivo and in vivo. Most prior in vivo editing techniques involved systemic injection of the editing machinery focused on targeting the liver, taking advantage of the efficiency of rAAV-mediated liver gene transfer. In this Example, a targeted delivery strategy of direct injection of rAAV encoding a transgene flanked by homology arms to initiate homology directed repair (HDR)-mediated gene editing of HSPCs in the bone marrow.

To obtain stable transgene expression without adversely affecting endogenous gene expression, gene editing was performed at a genomic safe harbor (GSH) site, AAVS1. First, the expression construct was tested in vitro to ensure optimal transgene expression in target cells. Expression of the reporter GFP expressed by the CMV. EF1a and MND promoters was evaluated.

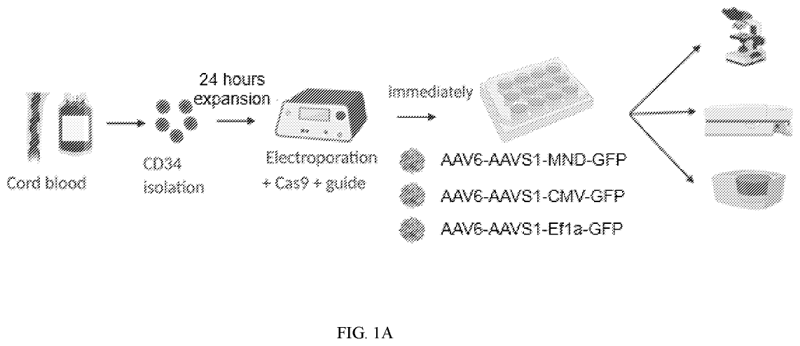

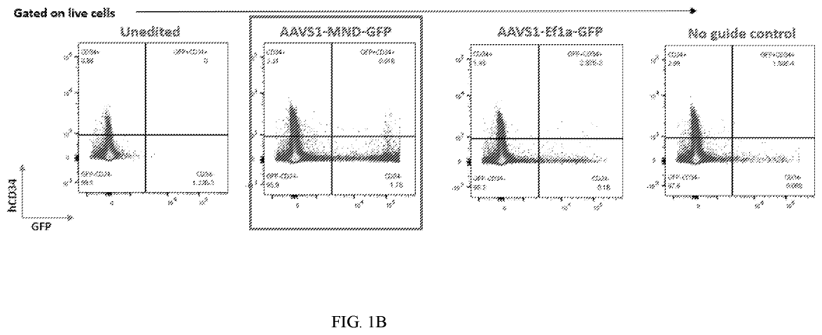

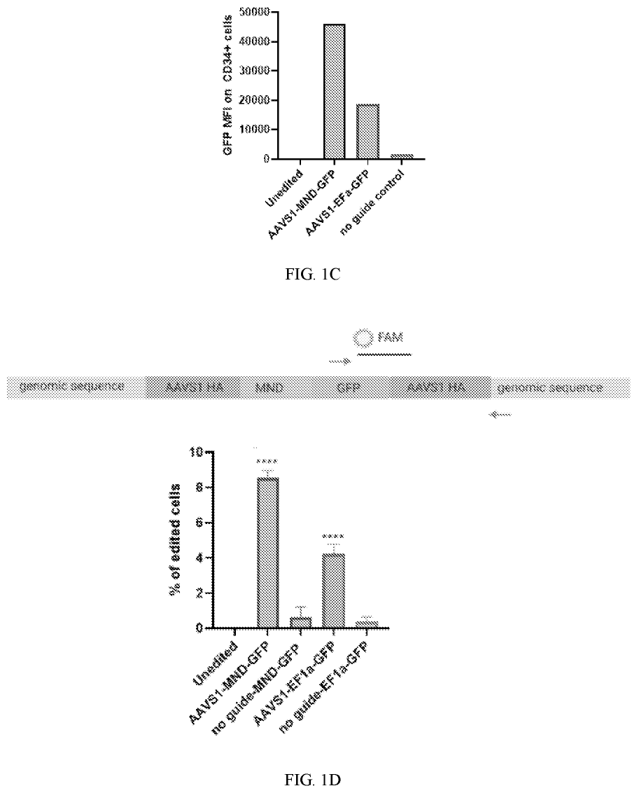

Human HSCs were isolated from cord blood by negative selection and enrichment of CD34+ cells, followed by electroporation with ribonucleoprotein complex containing AAVS1-specific guideRNA (gRNA) and Cas9. CD34+ cells were then transduced with rAAV6 expressing GFP from the different promoters (CMV, EF1a and MND) flanked by AAVS1 homology arms (FIG. 1A). The MND promoter reported robust and long-term (e.g., up to 9 days) GFP expression in human HSCs ex vivo. Additionally, the number of GFP expressing cells increased over time in culture, indicative of gene editing. When a transgene delivered to target HSCs using a rAAV6 capsid protein was integrated into the genome by homologous recombination (HR), the marked increase in transgene expression allows for identification of edited HSCs. A higher MFI of the GFP⁺ CD34⁺ cells was seen in the cells edited with the AAVS1-MND promoter construct compared to the Ef1a promoter and the no guide control (FIGS. 1B-1C). Further, the results were validated by digital droplet PCR assay designed for detecting HDR editing. CD34⁺ cells edited with AAVS1-MND-GFP showed higher percentage of editing and transgene (e.g., GFP) expression (FIG. 1D), which correlates with the imaging and flow cytometry data.

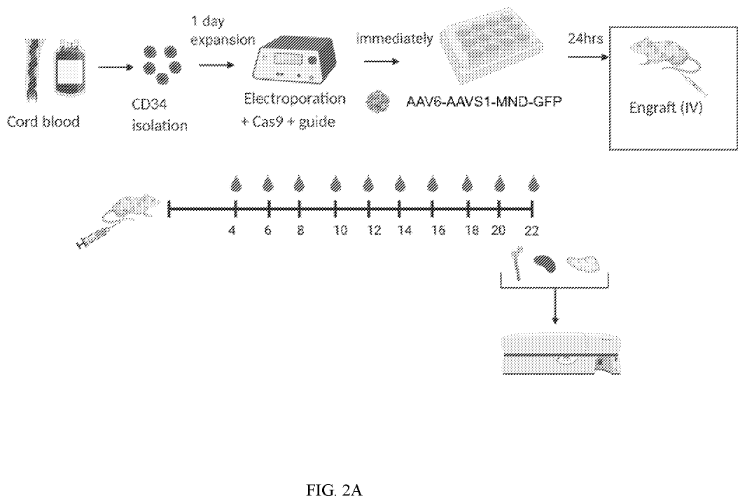

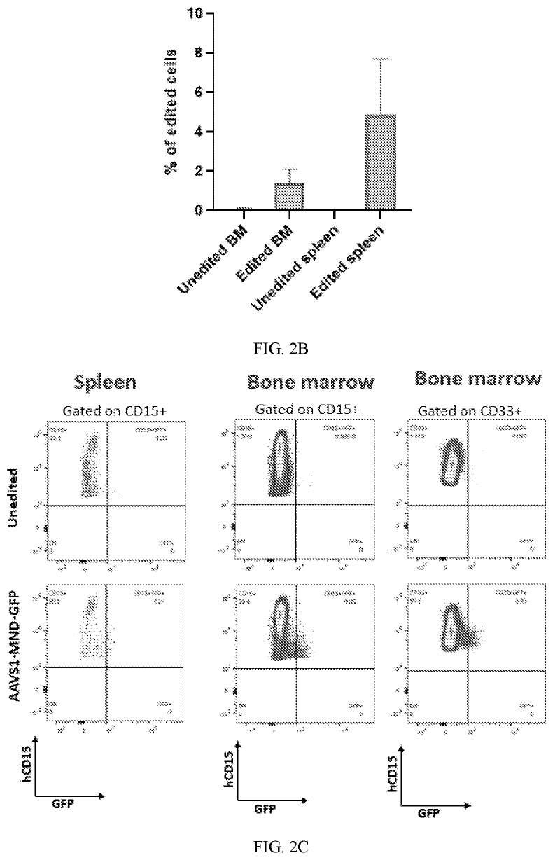

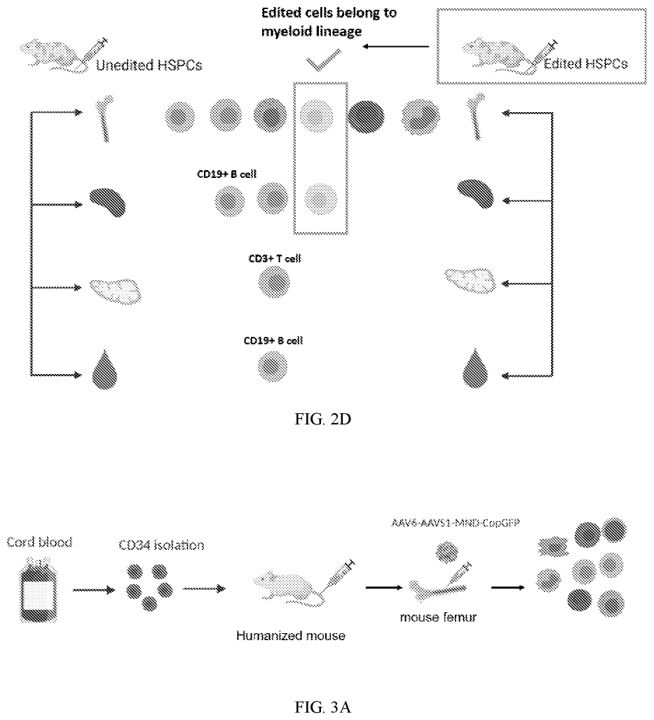

Based on the ex vivo results, the AAV construct having the MND promoter was used for subsequent in vivo experiments (FIG. 2A). In order to target HSCs and cells of the hematopoietic lineage, it was necessary to assess whether the MND promoter is expressed in these cells of interest. To test this in vivo, firstly conditions for optimum engraftment and differentiation of human CD34+ cells in the mouse bone marrow were established. Human HSCs were then electroporated with CRISPR/Cas editing machinery, and transduced with rAAV6 encoding GFP driven by MND promoter, flanked by AAVS1 homology arms, and engrafted into immunocompromised NBSGW (nonobese diabetic (NOD)-severe combined immunodeficiency (SCID)-gamma) mice. In this study, CD34⁺ cells were electroporated with the Cas9 and AAVS1 guide RNAs, followed by transduction with rAAV6-AAVS1-MND-GFP. The rAAV donor were injected into 4-6 weeks old mice recipient. The mice were bled to assess engraftment in the peripheral blood. The lineage composition of the engrafted cells was assessed at the terminal time point in the mouse bone marrow, spleen and thymus by flow cytometry (FIG. 2A). The results showed that successful human cell engraftment in the peripheral blood, bone marrow, spleen and thymus in both unedited and edited mice. Multilineage distribution of engrafted human cell population was observed. Human cells belonging to both myeloid and lymphoid lineage in the reconstituted NBSGW mice were observed. In order to check editing in these mice, genomic DNA from these humanized mice tissues were isolated and subjected to ddPCR. The mice that received engrafted edited cells showed editing (FIG. 2B). This data correlated with the flow cytometry data (FIG. 2C). FIG. 2D shows the distribution of human cell markers between unedited mouse and the mouse that received edited CD34+ cells. Both mice showed a good multilineage distribution in the BM, spleen, thymus and blood. The edited cells obtained from mice engrafted with edited cells are mostly myeloid cells (FIG. 2D).

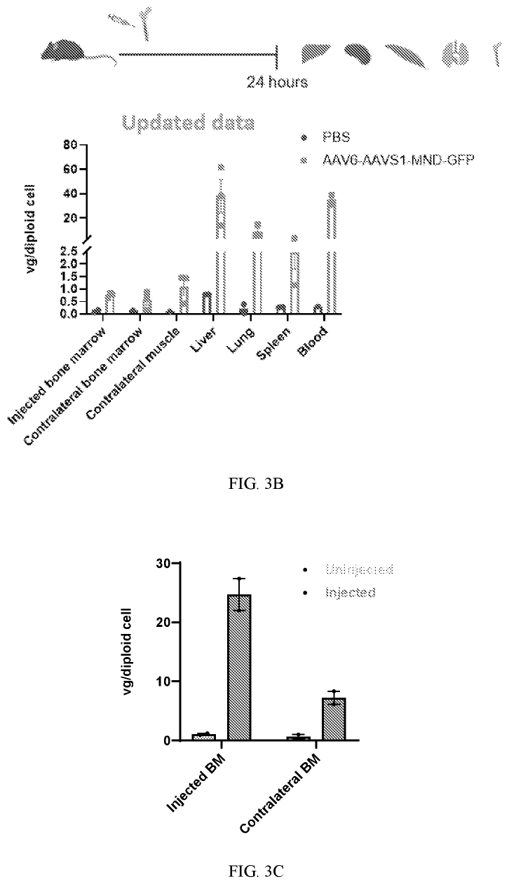

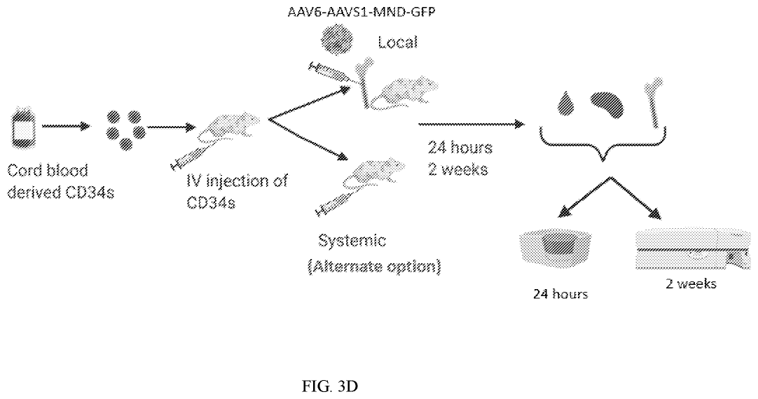

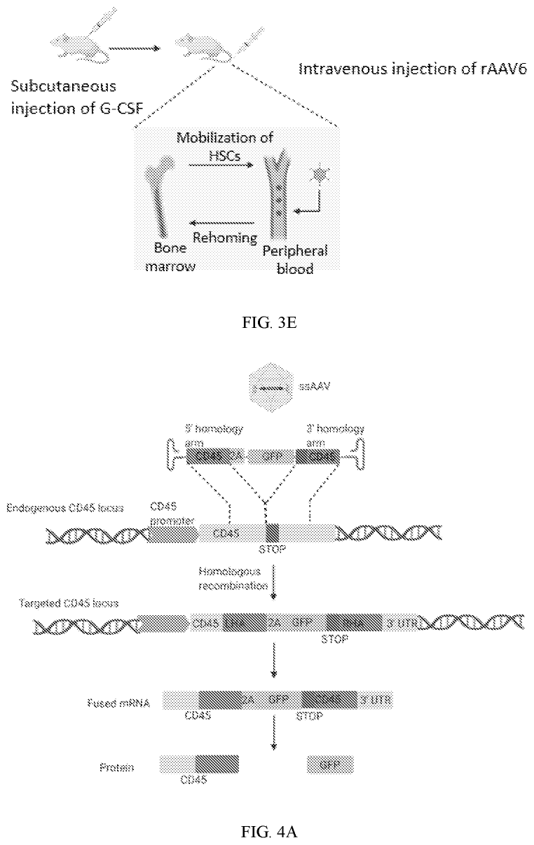

Next, to determine HDR-based editing efficiency in vivo, rAAV6 encoding AAVS1 homology arms and GFP driven by the MND promoter was injected directly into the bone marrow of engrafted NBSGW mice. Digital droplet PCT (ddPCR) results confirm that a localized intraosseous injection concentrates the vector in the targeted niche, thereby specifically targeting the bone marrow and enhancing transduction of the desired cell types. To test the biodistribution of the rAAV vectors by intraosseous injection, C57BL/6j mice were injected with rAAV6-AAVS1-MND-GFP via intraosseous injection (FIG. 3A). The biodistribution of the rAAV in different tissues was measured by ddPCR. The viral vectors were detected in the injected BM, liver, lungs, spleen, and blood. During the BM injection, some of the vectors ended up being in the ipsilateral and contralateral muscles as well. So, though it was expected that that the rAAV would reside in the BM, systemic distribution across various tissues was observed (FIG. 3B). Further, higher rAAV VG were observed by ddPCR in the engrafted cells in the injected BM as compared to the contralateral BM (FIG. 3C). Alternatively, systemic injection (e.g., intravenous injection) of rAAV6-AAVS1-MND-GFP is performed. HSCs can be mobilized from the BM into the peripheral blood. The rAAV administered by systemic injection transduces the mobilized HSCs in the peripheral blood. The HSCs then rehome to the bone marrow, thereby achieving in vivo editing of the HSCs (FIGS. 3D-3E).

### Example 2: Promoter Hijacking Approach for Cell Editing

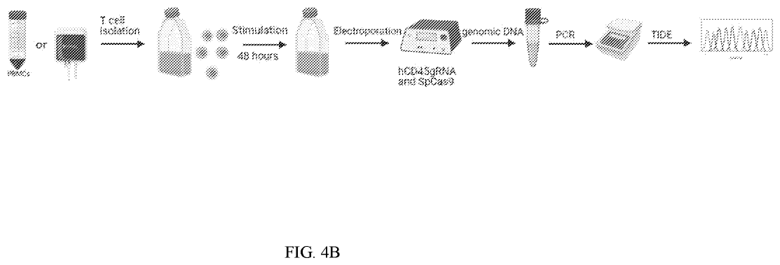

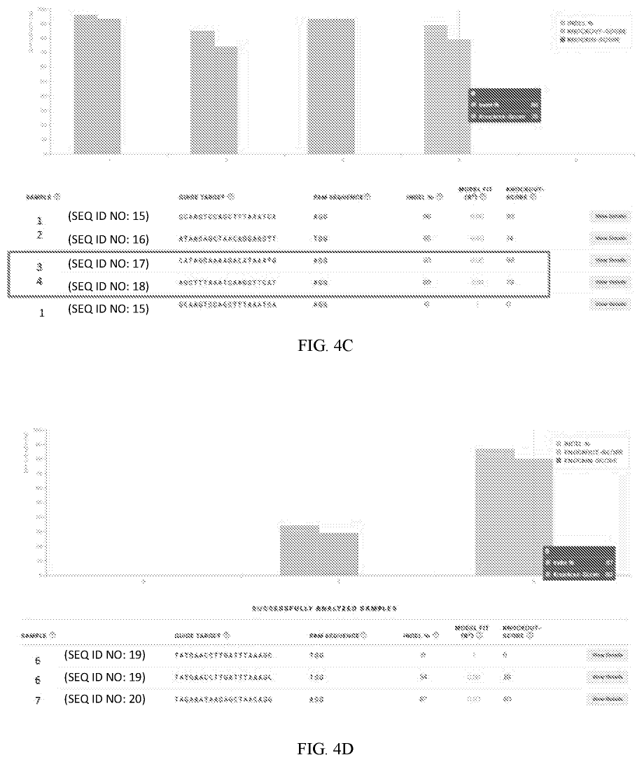

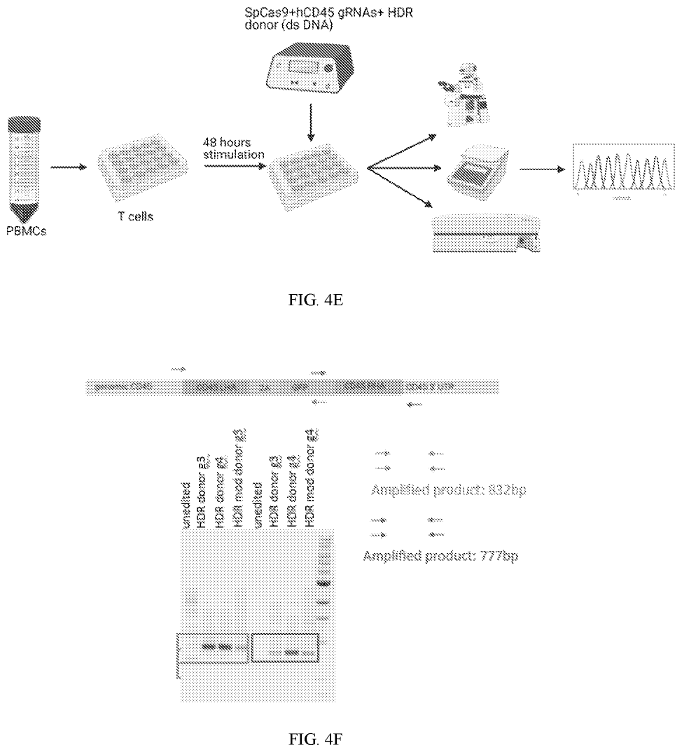

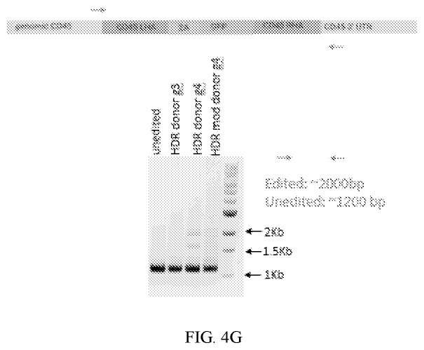

In this study, recombinant AAV vector containing a promoterless GFP coding sequence, preceded by the 2A peptide coding sequence and flanked by CD45 homology arms that covers the hCD45 stop codon in the 3′ homology arm was designed (CD45-T2A-GFP). The AAV vector was packaged into rAAV6 capsid protein. Homologous recombination resulted in integration of the designed AAV vector into the endogenous CD45 locus and generated a chimeric bicistronic mRNA, which was translated into two distinct proteins, CD45 and GFP due to the ribosomal skipping (see, e.g., Barzel, et al., Promoterless gene targeting without nucleases ameliorates haemophilia B in mice, Nature, 517 (2015), pp. 360-364) (FIG. 4A).

Seven gRNAs targeting human CD45 were tested for effectiveness in gene editing. T cell was isolated from peripheral blood and stimulated for 48 hours. Human CD45 gRNA and SpCas9 were delivered into the isolated T cells by electroporation. Genomic DNA was isolated and PCR was performed to test CD45 knockout score (FIG. 4B), which indicated the effectiveness of the gRNA. The results showed that gRNA 3 and 4 (SEQ ID NOs: 17 and 18) were high in knockout scores (FIGS. 4C-4D).

Next, in vitro editing of T cells using the CD45-T2A-GFP using gRNA3 and gRNA4 were tested. FIG. 4E shows the experimental design of T cell in vitro editing. hCD45 gRNA4 showed successful HDR (FIGS. 4F-4G).

### EQUIVALENTS

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an.” as used herein in the specification and in the claims, unless clearly indicated to the contrary, should be understood to mean “at least one.”

The phrase “and/or.” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of.” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising.” “including.” “carrying.” “having.” “containing,” “involving.” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

### SEQUENCE LISTING

The skilled artisan recognizes that certain sequences in the Sequence Listing are represented as linear nucleic acid sequences corresponding to circular plasmid sequences. Accordingly, in some embodiments, sequences described herein represent a contiguous polynucleotide (e.g., sequences sharing a continuous phosphate backbone), such that the first base and the last base of the linear representation are positioned next to one another. The Sequence listing contains the sequences as shown below:

>AAVS1 Homology arm (HA)-5′-
SEQ ID NO: 1
CAGAGCAGGGCCTTAGGGAAGCGGGACCCTGCTCTGGGCGGAGGAATATGTCCCAGATAGCACT
GGGGACTCTTTAAGGAAAGAAGGATGGAGAAAGAGAAAGGGAGTAGAGGCGGCCACGACCTGGT
GAACACCTAGGACGCACCATTCTCACAAAGGGAGTTTTCCACACGGACACCCCCCTCCTCACCA
CAGCCCTGCCAGGACGGGGCTGGCTACTGGCCTTATCTCACAGGTAAAACTGACGCACGGAGGA
ACAATATAAATTGGGGACTAGAAAGGTGAAGAGCCAAAGTTAGAACTCAGGACCAACTTATTCT
GATTTTGTTTTTCCAAACTGCTTCTCCTCTTGGGAAGTGTAAGGAAGCTGCAGCACCAGGATCA
GTGAAACGCACCAGACAGCCGCGTCAGAGCAGCTCAGGTTCTGGGAGAGGGTAGCGCAGGGTGG
CCACTGAGAACCGGGCAGGTCACGCATCCCCCCCTTCCCTCCCACCCCCTGCCAAGCTCTCCCT
CCCAGGATCCTCTCTGGC
>AAVS1 Homology arm (HA)-3′-
SEQ ID NO: 2
TGCTGTCCTGAAGTGGACATAGGGGCCCGGGTTGGAGGAAGAAGACTAGCTGAGCTCTCGGACC
CCTGGAAGATGCCATGACAGGGGGCTGGAAGAGCTAGCACAGACTAGAGAGGTAAGGGGGGTAG
GGGAGCTGCCCAAATGAAAGGAGTGAGAGGTGACCCGAATCCACAGGAGAACGGGGTGTCCAGG
CAAAGAAAGCAAGAGGATGGAGAGGTGGCTAAAGCCAGGGAGACGGGGTACTTTGGGGTTGTCC
AGAAAAACGGTGATGATGCAGGCCTACAAGAAGGGGAGGCGGGACGCAAGGGAGACATCCGTCG
GAGAAGGCCATCCTAAGAAACGAGAGATGGCACAGGCCCCAGAAGGAGAAGGAAAAGGGAACCC
AGCGAGTGAAGACGGCATGGGGTTGGGTGAGGGAGGAGAGATGCCCGGAGAGGACCCAGACACG
GGGAGGATCCGCTCAGAGGACATCACGTGGTGCAGCGCCGAGAAGGAAGTGCTC
>AAVS1-CMV-GFP vector-
SEQ ID NO: 3
CCTGCAGGCAGCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCCATC
ACTAGGGGTTCCTGCGGCCAGATCATCTAGACAGAGCAGGGCCTTAGGGAAGCGGGACCCTGCT
CTGGGCGGAGGAATATGTCCCAGATAGCACTGGGGACTCTTTAAGGAAAGAAGGATGGAGAAAG
AGAAAGGGAGTAGAGGCGGCCACGACCTGGTGAACACCTAGGACGCACCATTCTCACAAAGGGA
GTTTTCCACACGGACACCCCCCTCCTCACCACAGCCCTGCCAGGACGGGGCTGGCTACTGGCCT
TATCTCACAGGTAAAACTGACGCACGGAGGAACAATATAAATTGGGGACTAGAAAGGTGAAGAG
CCAAAGTTAGAACTCAGGACCAACTTATTCTGATTTTGTTTTTCCAAACTGCTTCTCCTCTTGG
GAAGTGTAAGGAAGCTGCAGCACCAGGATCAGTGAAACGCACCAGACAGCCGCGTCAGAGCAGC
TCAGGTTCTGGGAGAGGGTAGCGCAGGGTGGCCACTGAGAACCGGGCAGGTCACGCATCCCCCC
CTTCCCTCCCACCCCCTGCCAAGCTCTCCCTCCCAGGATCCTCTCTGGCTTCGAAGGCATTGAT
TATTGACTAGTTATTAATAGTAATCAATTACGGGGTCATTAGTTCATAGCCCATATATGGAGTT
CCGCGTTACATAACTTACGGTAAATGGCCCGCCTGGCTGACCGCCCAACGACCCCCGCCCATTG
ACGTCAATAATGACGTATGTTCCCATAGTAACGCCAATAGGGACTTTCCATTGACGTCAATGGG
TGGAGTATTTACGGTAAACTGCCCACTTGGCAGTACATCAAGTGTATCATATGCCAAGTCCGCC
CCCTATTGACGTCAATGACGGTAAATGGCCCGCCTGGCATTATGCCCAGTACATGACCTTACGG
GACTTTCCTACTTGGCAGTACATCTACGTATTAGTCATCGCTATTACCATGGTGATGCGGTTTT
GGCAGTACACCAATGGGCGTGGATAGCGGTTTGACTCACGGGGATTTCCAAGTCTCCACCCCAT
TGACGTCAATGGGAGTTTGTTTTGGCACCAAAATCAACGGGACTTTCCAAAATGTCGTAATAAC
CCCGCCCCGTTGACGCAAATGGGCGGTAGGCGTGTACGGTGGGAGGTCTATATAAGCAGAGGTC
GTTTAGTGAACCGTCAGATCACTAGTAGCTTTATTGCGGTAGTTTATCACAGTTAAATTGCTAA
CGCAGTCAGTGCTCGACTGATCACAGGTAAGTATCAAGGTTACAAGACAGGTTTAAGGAGGCCA
ATAGAAACTGGGCTTGTCGAGACAGAGAAGATTCTTGCGTTTCTGATAGGCACCTATTGGTCTT
ACTGACATCCACTTTGCCTTTCTCTCCACAGGGGTACCGAAGCCGCTAGCGCTACCGGTCGCCA
CCATGCCCGCCATGAAGATCGAGTGCCGCATCACCGGCACCCTGAACGGCGTGGAGTTCGAGCT
GGTGGGCGGCGGAGAGGGCACCCCCGAGCAGGGCCGCATGACCAACAAGATGAAGAGCACCAAA
GGCGCCCTGACCTTCAGCCCCTACCTGCTGAGCCACGTGATGGGCTACGGCTTCTACCACTTCG
GCACCTACCCCAGCGGCTACGAGAACCCCTTCCTGCACGCCATCAACAACGGCGGCTACACCAA
CACCCGCATCGAGAAGTACGAGGACGGCGGCGTGCTGCACGTGAGCTTCAGCTACCGCTACGAG
GCCGGCCGCGTGATCGGCGACTTCAAGGTGGTGGGCACCGGCTTCCCCGAGGACAGCGTGATCT
TCACCGACAAGATCATCCGCAGCAACGCCACCGTGGAGCACCTGCACCCCATGGGCGATAACGT
GCTGGTGGGCAGCTTCGCCCGCACCTTCAGCCTGCGCGACGGCGGCTACTACAGCTTCGTGGTG
GACAGCCACATGCACTTCAAGAGCGCCATCCACCCCAGCATCCTGCAGAACGGGGGCCCCATGT
TCGCCTTCCGCCGCGTGGAGGAGCTGCACAGCAACACCGAGCTGGGCATCGTGGAGTACCAGCA
CGCCTTCAAGACCCCCATCGCCTTCGCCAGATCTCGAGCTCGATGAGTTTGGACAAACCACAAC
TAGAATGCAGTGAAAAAAATGCTTTATTTGTGAAATTTGTGATGCTATTGCTTTATTTGTAACC
ATTATAAGCTGCAATAAACAAGTTACGCGTTGCTGTCCTGAAGTGGACATAGGGGCCCGGGTTG
GAGGAAGAAGACTAGCTGAGCTCTCGGACCCCTGGAAGATGCCATGACAGGGGGCTGGAAGAGC
TAGCACAGACTAGAGAGGTAAGGGGGGTAGGGGAGCTGCCCAAATGAAAGGAGTGAGAGGTGAC
CCGAATCCACAGGAGAACGGGGTGTCCAGGCAAAGAAAGCAAGAGGATGGAGAGGTGGCTAAAG
CCAGGGAGACGGGGTACTTTGGGGTTGTCCAGAAAAACGGTGATGATGCAGGCCTACAAGAAGG
GGAGGCGGGACGCAAGGGAGACATCCGTCGGAGAAGGCCATCCTAAGAAACGAGAGATGGCACA
GGCCCCAGAAGGAGAAGGAAAAGGGAACCCAGCGAGTGAAGACGGCATGGGGTTGGGTGAGGGA
GGAGAGATGCCCGGAGAGGACCCAGACACGGGGAGGATCCGCTCAGAGGACATCACGTGGTGCA
GCGCCGAGAAGGAAGTGCTCATCGATAGGCCGCAGGAACCCCTAGTGATGGAGTTGGCCACTCC
CTCTCTGCGCGCTCGCTCGCTCACTGAGGCCGGGCGACCAAAGGTCGCCCGACGCCCGGGCGGC
CTCAGTGAGCGAGCGAGCGCGCAGCTGCCTGCAGGACATGTGAGCAAAAGGCCAGCAAAAGGCC
AGGAACCGTAAAAAGGCCGCGTTGCTGGCGTTTTTCCATAGGCTCCGCCCCCCTGACGAGCATC
ACAAAAATCGACGCTCAAGTCAGAGGTGGCGAAACCCGACAGGACTATAAAGATACCAGGCGTT
TCCCCCTGGAAGCTCCCTCGTGCGCTCTCCTGTTCCGACCCTGCCGCTTACCGGATACCTGTCC
GCCTTTCTCCCTTCGGGAAGCGTGGCGCTTTCTCATAGCTCACGCTGTAGGTATCTCAGTTCGG
TGTAGGTCGTTCGCTCCAAGCTGGGCTGTGTGCACGAACCCCCCGTTCAGCCCGACCGCTGCGC
CTTATCCGGTAACTATCGTCTTGAGTCCAACCCGGTAAGACACGACTTATCGCCACTGGCAGCA
GCCACTGGTAACAGGATTAGCAGAGCGAGGTATGTAGGCGGTGCTACAGAGTTCTTGAAGTGGT
GGCCTAACTACGGCTACACTAGAAGAACAGTATTTGGTATCTGCGCTCTGCTGAAGCCAGTTAC
CTTCGGAAAAAGAGTTGGTAGCTCTTGATCCGGCAAACAAACCACCGCTGGTAGCGGTGGTTTT
TTTGTTTGCAAGCAGCAGATTACGCGCAGAAAAAAAGGATCTCAAGAAGATCCTTTGATCTTTT
CTACGGGGTCTGACGCTCAGTGGAACGAAAACTCACGTTAAGGGATTTTGGTCATGAGATTATC
AAAAAGGATCTTCACCTAGATCCTTTTAAATTAAAAATGAAGTTTTAAATCAATCTAAAGTATA
TATGAGTAAACTTGGTCTGACAGTTACCAATGCTTAATCAGTGAGGCACCTATCTCAGCGATCT
GTCTATTTCGTTCATCCATAGTTGCCTGACTCCCCGTCGTGTAGATAACTACGATACGGGAGGG
CTTACCATCTGGCCCCAGTGCTGCAATGATACCGCGAGACCCACGCTCACCGGCTCCAGATTTA
TCAGCAATAAACCAGCCAGCCGGAAGGGCCGAGCGCAGAAGTGGTCCTGCAACTTTATCCGCCT
CCATCCAGTCTATTAATTGTTGCCGGGAAGCTAGAGTAAGTAGTTCGCCAGTTAATAGTTTGCG
CAACGTTGTTGCCATTGCTACAGGCATCGTGGTGTCACGCTCGTCGTTTGGTATGGCTTCATTC
AGCTCCGGTTCCCAACGATCAAGGCGAGTTACATGATCCCCCATGTTGTGCAAAAAAGCGGTTA
GCTCCTTCGGTCCTCCGATCGTTGTCAGAAGTAAGTTGGCCGCAGTGTTATCACTCATGGTTAT
GGCAGCACTGCATAATTCTCTTACTGTCATGCCATCCGTAAGATGCTTTTCTGTGACTGGTGAG
TACTCAACCAAGTCATTCTGAGAATAGTGTATGCGGCGACCGAGTTGCTCTTGCCCGGCGTCAA
TACGGGATAATACCGCGCCACATAGCAGAACTTTAAAAGTGCTCATCATTGGAAAACGTTCTTC
GGGGCGAAAACTCTCAAGGATCTTACCGCTGTTGAGATCCAGTTCGATGTAACCCACTCGTGCA
CCCAACTGATCTTCAGCATCTTTTACTTTCACCAGCGTTTCTGGGTGAGCAAAAACAGGAAGGC
AAAATGCCGCAAAAAAGGGAATAAGGGCGACACGGAAATGTTGAATACTCATACTCTTCCTTTT
TCAATATTATTGAAGCATTTATCAGGGTTATTGTCTCATGAGCGGATACATATTTGAATGTATT
TAGAAAAATAAACAAATAGGGGTTCCGCGCACATTTCCCCGAAAAGTGCCACCTGACGTCTAAG
AAACCATTATTATCATGACATTAACCTATAAAAATAGGCGTATCACGAGGCCCTTTCGTCTCGC
GCGTTTCGGTGATGACGGTGAAAACCTCTGACACATGCAGCTCCCGGAGACGGTCACAGCTTGT
CTGTAAGCGGATGCCGGGAGCAGACAAGCCCGTCAGGGCGCGTCAGCGGGTGTTGGCGGGTGTC
GGGGCTGGCTTAACTATGCGGCATCAGAGCAGATTGTACTGAGAGTGCACCATAAAATTGTAAA
CGTTAATATTTTGTTAAAATTCGCGTTAAATTTTTGTTAAATCAGCTCATTTTTTAACCAATAG
ACCGAAATCGGCAAAATCCCTTATAAATCAAAAGAATAGCCCGAGATAGAGTTGAGTGTTGTTC
CAGTTTGGAACAAGAGTCCACTATTAAAGAACGTGGACTCCAACGTCAAAGGGCGAAAAACCGT
CTATCAGGGCGATGGCCCACTACGTGAACCATCACCCAAATCAAGTTTTTTGGGGTCGAGGTGC
CGTAAAGCACTAAATCGGAACCCTAAAGGGAGCCCCCGATTTAGAGCTTGACGGGGAAAGCCGG
CGAACGTGGCGAGAAAGGAAGGGAAGAAAGCGAAAGGAGCGGGCGCTAAGGCGCTGGCAAGTGT
AGCGGTCACGCTGCGCGTAACCACCACACCCGCCGCGCTTAATGCGCCGCTACAGGGCGCGTAC
TATGGTTGCTTTGACGTATGCGGTGTGAAATACCGCACAGATGCGTAAGGAGAAAATACCGCAT
CAGGCGCC
>AAVS1-MND-GFP vector-
SEQ ID NO: 4
CCTGCAGGCAGCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCCATC
ACTAGGGGTTCCTGCGGCCAGATCATCTAGACAGAGCAGGGCCTTAGGGAAGCGGGACCCTGCT
CTGGGCGGAGGAATATGTCCCAGATAGCACTGGGGACTCTTTAAGGAAAGAAGGATGGAGAAAG
AGAAAGGGAGTAGAGGCGGCCACGACCTGGTGAACACCTAGGACGCACCATTCTCACAAAGGGA
GTTTTCCACACGGACACCCCCCTCCTCACCACAGCCCTGCCAGGACGGGGCTGGCTACTGGCCT
TATCTCACAGGTAAAACTGACGCACGGAGGAACAATATAAATTGGGGACTAGAAAGGTGAAGAG
CCAAAGTTAGAACTCAGGACCAACTTATTCTGATTTTGTTTTTCCAAACTGCTTCTCCTCTTGG
GAAGTGTAAGGAAGCTGCAGCACCAGGATCAGTGAAACGCACCAGACAGCCGCGTCAGAGCAGC
TCAGGTTCTGGGAGAGGGTAGCGCAGGGTGGCCACTGAGAACCGGGCAGGTCACGCATCCCCCC
CTTCCCTCCCACCCCCTGCCAAGCTCTCCCTCCCAGGATCCTCTCTGGCTTCGAAATCGATCAC
GAGACTAGCCTCGAGAAGCTTGATGGCCGCCAGTGTGATGGATATCTGCAGAATTCGCCCTTAT
GGGGATCCGAACAGAGAGACAGCAGAATATGGGCCAAACAGGATATCTGTGGTAAGCAGTTCCT
GCCCCGGCTCAGGGCCAAGAACAGTTGGAACAGCAGAATATGGGCCAAACAGGATATCTGTGGT
AAGCAGTTCCTGCCCCGGCTCAGGGCCAAGAACAGATGGTCCCCAGATGCGGTCCCGCCCTCAG
CAGTTTCTAGAGAACCATCAGATGTTTCCAGGGTGCCCCAAGGACCTGAAATGACCCTGTGCCT
TATTTGAACTAACCAATCAGTTCGCTTCTCGCTTCTGTTCGCGCGCTTCTGCTCCCCGAGCTCT
ATATAAGCAGAGCTCGTTTAGTGAACCGTCAGATCGCCTGGAGACGCCATCCACGCTGTTTTGA
CCTCCATAGAAGACACCGACTCTAGAGGATCCACCGGTCGCCACCATGCCCGCCATGAAGATCG
AGTGCCGCATCACCGGCACCCTGAACGGCGTGGAGTTCGAGCTGGTGGGCGGCGGAGAGGGCAC
CCCCGAGCAGGGCCGCATGACCAACAAGATGAAGAGCACCAAAGGCGCCCTGACCTTCAGCCCC
TACCTGCTGAGCCACGTGATGGGCTACGGCTTCTACCACTTCGGCACCTACCCCAGCGGCTACG
AGAACCCCTTCCTGCACGCCATCAACAACGGCGGCTACACCAACACCCGCATCGAGAAGTACGA
GGACGGCGGCGTGCTGCACGTGAGCTTCAGCTACCGCTACGAGGCCGGCCGCGTGATCGGCGAC
TTCAAGGTGGTGGGCACCGGCTTCCCCGAGGACAGCGTGATCTTCACCGACAAGATCATCCGCA
GCAACGCCACCGTGGAGCACCTGCACCCCATGGGCGATAACGTGCTGGTGGGCAGCTTCGCCCG
CACCTTCAGCCTGCGCGACGGCGGCTACTACAGCTTCGTGGTGGACAGCCACATGCACTTCAAG
AGCGCCATCCACCCCAGCATCCTGCAGAACGGGGGCCCCATGTTCGCCTTCCGCCGCGTGGAGG
AGCTGCACAGCAACACCGAGCTGGGCATCGTGGAGTACCAGCACGCCTTCAAGACCCCCATCGC
CTTCGCCAGATCTCGAGCTCGATGAGTTTGGACAAACCACAACTAGAATGCAGTGAAAAAAATG
CTTTATTTGTGAAATTTGTGATGCTATTGCTTTATTTGTAACCATTATAAGCTGCAATAAACAA
GTTACGCGTTGCTGTCCTGAAGTGGACATAGGGGCCCGGGTTGGAGGAAGAAGACTAGCTGAGC
TCTCGGACCCCTGGAAGATGCCATGACAGGGGGCTGGAAGAGCTAGCACAGACTAGAGAGGTAA
GGGGGGTAGGGGAGCTGCCCAAATGAAAGGAGTGAGAGGTGACCCGAATCCACAGGAGAACGGG
GTGTCCAGGCAAAGAAAGCAAGAGGATGGAGAGGTGGCTAAAGCCAGGGAGACGGGGTACTTTG
GGGTTGTCCAGAAAAACGGTGATGATGCAGGCCTACAAGAAGGGGAGGCGGGACGCAAGGGAGA
CATCCGTCGGAGAAGGCCATCCTAAGAAACGAGAGATGGCACAGGCCCCAGAAGGAGAAGGAAA
AGGGAACCCAGCGAGTGAAGACGGCATGGGGTTGGGTGAGGGAGGAGAGATGCCCGGAGAGGAC
CCAGACACGGGGAGGATCCGCTCAGAGGACATCACGTGGTGCAGCGCCGAGAAGGAAGTGCTCA
TCGATAGGCCGCAGGAACCCCTAGTGATGGAGTTGGCCACTCCCTCTCTGCGCGCTCGCTCGCT
CACTGAGGCCGGGCGACCAAAGGTCGCCCGACGCCCGGGCGGCCTCAGTGAGCGAGCGAGCGCG
CAGCTGCCTGCAGGACATGTGAGCAAAAGGCCAGCAAAAGGCCAGGAACCGTAAAAAGGCCGCG
TTGCTGGCGTTTTTCCATAGGCTCCGCCCCCCTGACGAGCATCACAAAAATCGACGCTCAAGTC
AGAGGTGGCGAAACCCGACAGGACTATAAAGATACCAGGCGTTTCCCCCTGGAAGCTCCCTCGT
GCGCTCTCCTGTTCCGACCCTGCCGCTTACCGGATACCTGTCCGCCTTTCTCCCTTCGGGAAGC
GTGGCGCTTTCTCATAGCTCACGCTGTAGGTATCTCAGTTCGGTGTAGGTCGTTCGCTCCAAGC
TGGGCTGTGTGCACGAACCCCCCGTTCAGCCCGACCGCTGCGCCTTATCCGGTAACTATCGTCT
TGAGTCCAACCCGGTAAGACACGACTTATCGCCACTGGCAGCAGCCACTGGTAACAGGATTAGC
AGAGCGAGGTATGTAGGCGGTGCTACAGAGTTCTTGAAGTGGTGGCCTAACTACGGCTACACTA
GAAGAACAGTATTTGGTATCTGCGCTCTGCTGAAGCCAGTTACCTTCGGAAAAAGAGTTGGTAG
CTCTTGATCCGGCAAACAAACCACCGCTGGTAGCGGTGGTTTTTTTGTTTGCAAGCAGCAGATT
ACGCGCAGAAAAAAAGGATCTCAAGAAGATCCTTTGATCTTTTCTACGGGGTCTGACGCTCAGT
GGAACGAAAACTCACGTTAAGGGATTTTGGTCATGAGATTATCAAAAAGGATCTTCACCTAGAT
CCTTTTAAATTAAAAATGAAGTTTTAAATCAATCTAAAGTATATATGAGTAAACTTGGTCTGAC
AGTTACCAATGCTTAATCAGTGAGGCACCTATCTCAGCGATCTGTCTATTTCGTTCATCCATAG
TTGCCTGACTCCCCGTCGTGTAGATAACTACGATACGGGAGGGCTTACCATCTGGCCCCAGTGC
TGCAATGATACCGCGAGACCCACGCTCACCGGCTCCAGATTTATCAGCAATAAACCAGCCAGCC
GGAAGGGCCGAGCGCAGAAGTGGTCCTGCAACTTTATCCGCCTCCATCCAGTCTATTAATTGTT
GCCGGGAAGCTAGAGTAAGTAGTTCGCCAGTTAATAGTTTGCGCAACGTTGTTGCCATTGCTAC
AGGCATCGTGGTGTCACGCTCGTCGTTTGGTATGGCTTCATTCAGCTCCGGTTCCCAACGATCA
AGGCGAGTTACATGATCCCCCATGTTGTGCAAAAAAGCGGTTAGCTCCTTCGGTCCTCCGATCG
TTGTCAGAAGTAAGTTGGCCGCAGTGTTATCACTCATGGTTATGGCAGCACTGCATAATTCTCT
TACTGTCATGCCATCCGTAAGATGCTTTTCTGTGACTGGTGAGTACTCAACCAAGTCATTCTGA
GAATAGTGTATGCGGCGACCGAGTTGCTCTTGCCCGGCGTCAATACGGGATAATACCGCGCCAC
ATAGCAGAACTTTAAAAGTGCTCATCATTGGAAAACGTTCTTCGGGGCGAAAACTCTCAAGGAT
CTTACCGCTGTTGAGATCCAGTTCGATGTAACCCACTCGTGCACCCAACTGATCTTCAGCATCT
TTTACTTTCACCAGCGTTTCTGGGTGAGCAAAAACAGGAAGGCAAAATGCCGCAAAAAAGGGAA
TAAGGGCGACACGGAAATGTTGAATACTCATACTCTTCCTTTTTCAATATTATTGAAGCATTTA
TCAGGGTTATTGTCTCATGAGCGGATACATATTTGAATGTATTTAGAAAAATAAACAAATAGGG
GTTCCGCGCACATTTCCCCGAAAAGTGCCACCTGACGTCTAAGAAACCATTATTATCATGACAT
TAACCTATAAAAATAGGCGTATCACGAGGCCCTTTCGTCTCGCGCGTTTCGGTGATGACGGTGA
AAACCTCTGACACATGCAGCTCCCGGAGACGGTCACAGCTTGTCTGTAAGCGGATGCCGGGAGC
AGACAAGCCCGTCAGGGCGCGTCAGCGGGTGTTGGCGGGTGTCGGGGCTGGCTTAACTATGCGG
CATCAGAGCAGATTGTACTGAGAGTGCACCATAAAATTGTAAACGTTAATATTTTGTTAAAATT
CGCGTTAAATTTTTGTTAAATCAGCTCATTTTTTAACCAATAGACCGAAATCGGCAAAATCCCT
TATAAATCAAAAGAATAGCCCGAGATAGAGTTGAGTGTTGTTCCAGTTTGGAACAAGAGTCCAC
TATTAAAGAACGTGGACTCCAACGTCAAAGGGCGAAAAACCGTCTATCAGGGCGATGGCCCACT
ACGTGAACCATCACCCAAATCAAGTTTTTTGGGGTCGAGGTGCCGTAAAGCACTAAATCGGAAC
CCTAAAGGGAGCCCCCGATTTAGAGCTTGACGGGGAAAGCCGGCGAACGTGGCGAGAAAGGAAG
GGAAGAAAGCGAAAGGAGCGGGCGCTAAGGCGCTGGCAAGTGTAGCGGTCACGCTGCGCGTAAC
CACCACACCCGCCGCGCTTAATGCGCCGCTACAGGGCGCGTACTATGGTTGCTTTGACGTATGC
GGTGTGAAATACCGCACAGATGCGTAAGGAGAAAATACCGCATCAGGCGCC
>AAVS1-EF1a-GFP vector-
SEQ ID NO: 5
CCTGCAGGCAGCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCCATC
ACTAGGGGTTCCTGCGGCCAGATCATCTAGACAGAGCAGGGCCTTAGGGAAGCGGGACCCTGCT
CTGGGCGGAGGAATATGTCCCAGATAGCACTGGGGACTCTTTAAGGAAAGAAGGATGGAGAAAG
AGAAAGGGAGTAGAGGCGGCCACGACCTGGTGAACACCTAGGACGCACCATTCTCACAAAGGGA
GTTTTCCACACGGACACCCCCCTCCTCACCACAGCCCTGCCAGGACGGGGCTGGCTACTGGCCT
TATCTCACAGGTAAAACTGACGCACGGAGGAACAATATAAATTGGGGACTAGAAAGGTGAAGAG
CCAAAGTTAGAACTCAGGACCAACTTATTCTGATTTTGTTTTTCCAAACTGCTTCTCCTCTTGG
GAAGTGTAAGGAAGCTGCAGCACCAGGATCAGTGAAACGCACCAGACAGCCGCGTCAGAGCAGC
TCAGGTTCTGGGAGAGGGTAGCGCAGGGTGGCCACTGAGAACCGGGCAGGTCACGCATCCCCCC
CTTCCCTCCCACCCCCTGCCAAGCTCTCCCTCCCAGGATCCTCTCTGGCTTCGAAGCTCCGGTG
CCCGTCAGTGGGCAGAGCGCACATCGCCCACAGTCCCCGAGAAGTTGGGGGGAGGGGTCGGCAA
TTGAACCGGTGCCTAGAGAAGGTGGCGCGGGGTAAACTGGGAAAGTGATGTCGTGTACTGGCTC
CGCCTTTTTCCCGAGGGTGGGGGAGAACCGTATATAAGTGCAGTAGTCGCCGTGAACGTTCTTT
TTCGCAACGGGTTTGCCGCCAGAACACAGGTAAGTGCCGTGTGTGGTTCCCGCGGGCCTGGCCT
CTTTACGGGTTATGGCCCTTGCGTGCCTTGAATTACTTCCACGCCCCTGGCTGCAGTACGTGAT
TCTTGATCCCGAGCTTCGGGTTGGAAGTGGGTGGGAGAGTTCGAGGCCTTGCGCTTAAGGAGCC
CCTTCGCCTCGTGCTTGAGTTGAGGCCTGGCCTGGGCGCTGGGGCCGCCGCGTGCGAATCTGGT
GGCACCTTCGCGCCTGTCTCGCTGCTTTCGATAAGTCTCTAGCCATTTAAAATTTTTGATGACC
TGCTGCGACGCTTTTTTTCTGGCAAGATAGTCTTGTAAATGCGGGCCAAGATCTGCACACTGGT
ATTTCGGTTTTTGGGGCCGCGGGCGGCGACGGGGCCCGTGCGTCCCAGCGCACATGTTCGGCGA
GGCGGGGCCTGCGAGCGCGGCCACCGAGAATCGGACGGGGGTAGTCTCAAGCTGGCCGGCCTGC
TCTGGTGCCTGGCCTCGCGCCGCCGTGTATCGCCCCGCCCTGGGCGGCAAGGCTGGCCCGGTCG
GCACCAGTTGCGTGAGCGGAAAGATGGCCGCTTCCCGGCCCTGCTGCAGGGAGCTCAAAATGGA
GGACGCGGCGCTCGGGAGAGCGGGCGGGTGAGTCACCCACACAAAGGAAAAGGGCCTTTCCGTC
CTCAGCCGTCGCTTCATGTGACTCCACGGAGTACCGGGCGCCGTCCAGGCACCTCGATTAGTTC
TCGAGCTTTTGGAGTACGTCGTCTTTAGGTTGGGGGGAGGGGTTTTATGCGATGGAGTTTCCCC
ACACTGAGTGGGTGGAGACTGAAGTTAGGCCAGCTTGGCACTTGATGTAATTCTCCTTGGAATT
TGCCCTTTTTGAGTTTGGATCTTGGTTCATTCTCAAGCCTCAGACAGTGGTTCAAAGTTTTTTT
CTTCCATTTCAGGTGTCGTGAACCGGTCGCCACCATGCCCGCCATGAAGATCGAGTGCCGCATC
ACCGGCACCCTGAACGGCGTGGAGTTCGAGCTGGTGGGCGGCGGAGAGGGCACCCCCGAGCAGG
GCCGCATGACCAACAAGATGAAGAGCACCAAAGGCGCCCTGACCTTCAGCCCCTACCTGCTGAG
CCACGTGATGGGCTACGGCTTCTACCACTTCGGCACCTACCCCAGCGGCTACGAGAACCCCTTC
CTGCACGCCATCAACAACGGCGGCTACACCAACACCCGCATCGAGAAGTACGAGGACGGCGGCG
TGCTGCACGTGAGCTTCAGCTACCGCTACGAGGCCGGCCGCGTGATCGGCGACTTCAAGGTGGT
GGGCACCGGCTTCCCCGAGGACAGCGTGATCTTCACCGACAAGATCATCCGCAGCAACGCCACC
GTGGAGCACCTGCACCCCATGGGCGATAACGTGCTGGTGGGCAGCTTCGCCCGCACCTTCAGCC
TGCGCGACGGCGGCTACTACAGCTTCGTGGTGGACAGCCACATGCACTTCAAGAGCGCCATCCA
CCCCAGCATCCTGCAGAACGGGGGCCCCATGTTCGCCTTCCGCCGCGTGGAGGAGCTGCACAGC
AACACCGAGCTGGGCATCGTGGAGTACCAGCACGCCTTCAAGACCCCCATCGCCTTCGCCAGAT
CTCGAGCTCGATGAGTTTGGACAAACCACAACTAGAATGCAGTGAAAAAAATGCTTTATTTGTG
AAATTTGTGATGCTATTGCTTTATTTGTAACCATTATAAGCTGCAATAAACAAGTTACGCGTTG
CTGTCCTGAAGTGGACATAGGGGCCCGGGTTGGAGGAAGAAGACTAGCTGAGCTCTCGGACCCC
TGGAAGATGCCATGACAGGGGGCTGGAAGAGCTAGCACAGACTAGAGAGGTAAGGGGGGTAGGG
GAGCTGCCCAAATGAAAGGAGTGAGAGGTGACCCGAATCCACAGGAGAACGGGGTGTCCAGGCA
AAGAAAGCAAGAGGATGGAGAGGTGGCTAAAGCCAGGGAGACGGGGTACTTTGGGGTTGTCCAG
AAAAACGGTGATGATGCAGGCCTACAAGAAGGGGAGGCGGGACGCAAGGGAGACATCCGTCGGA
GAAGGCCATCCTAAGAAACGAGAGATGGCACAGGCCCCAGAAGGAGAAGGAAAAGGGAACCCAG
CGAGTGAAGACGGCATGGGGTTGGGTGAGGGAGGAGAGATGCCCGGAGAGGACCCAGACACGGG
GAGGATCCGCTCAGAGGACATCACGTGGTGCAGCGCCGAGAAGGAAGTGCTCATCGATAGGCCG
CAGGAACCCCTAGTGATGGAGTTGGCCACTCCCTCTCTGCGCGCTCGCTCGCTCACTGAGGCCG
GGCGACCAAAGGTCGCCCGACGCCCGGGCGGCCTCAGTGAGCGAGCGAGCGCGCAGCTGCCTGC
AGGACATGTGAGCAAAAGGCCAGCAAAAGGCCAGGAACCGTAAAAAGGCCGCGTTGCTGGCGTT
TTTCCATAGGCTCCGCCCCCCTGACGAGCATCACAAAAATCGACGCTCAAGTCAGAGGTGGCGA
AACCCGACAGGACTATAAAGATACCAGGCGTTTCCCCCTGGAAGCTCCCTCGTGCGCTCTCCTG
TTCCGACCCTGCCGCTTACCGGATACCTGTCCGCCTTTCTCCCTTCGGGAAGCGTGGCGCTTTC
TCATAGCTCACGCTGTAGGTATCTCAGTTCGGTGTAGGTCGTTCGCTCCAAGCTGGGCTGTGTG
CACGAACCCCCCGTTCAGCCCGACCGCTGCGCCTTATCCGGTAACTATCGTCTTGAGTCCAACC
CGGTAAGACACGACTTATCGCCACTGGCAGCAGCCACTGGTAACAGGATTAGCAGAGCGAGGTA
TGTAGGCGGTGCTACAGAGTTCTTGAAGTGGTGGCCTAACTACGGCTACACTAGAAGAACAGTA
TTTGGTATCTGCGCTCTGCTGAAGCCAGTTACCTTCGGAAAAAGAGTTGGTAGCTCTTGATCCG
GCAAACAAACCACCGCTGGTAGCGGTGGTTTTTTTGTTTGCAAGCAGCAGATTACGCGCAGAAA
AAAAGGATCTCAAGAAGATCCTTTGATCTTTTCTACGGGGTCTGACGCTCAGTGGAACGAAAAC
TCACGTTAAGGGATTTTGGTCATGAGATTATCAAAAAGGATCTTCACCTAGATCCTTTTAAATT
AAAAATGAAGTTTTAAATCAATCTAAAGTATATATGAGTAAACTTGGTCTGACAGTTACCAATG
CTTAATCAGTGAGGCACCTATCTCAGCGATCTGTCTATTTCGTTCATCCATAGTTGCCTGACTC
CCCGTCGTGTAGATAACTACGATACGGGAGGGCTTACCATCTGGCCCCAGTGCTGCAATGATAC
CGCGAGACCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGGGAAGCT
AGAGTAAGTAGTTCGCCAGTTAATAGTTTGCGCAACGTTGTTGCCATTGCTACAGGCATCGTGG
TGTCACGCTCGTCGTTTGGTATGGCTTCATTCAGCTCCGGTTCCCAACGATCAAGGCGAGTTAC
ATGATCCCCCATGTTGTGCAAAAAAGCGGTTAGCTCCTTCGGTCCTCCGATCGTTGTCAGAAGT
AAGTTGGCCGCAGTGTTATCACTCATGGTTATGGCAGCACTGCATAATTCTCTTACTGTCATGC
CATCCGTAAGATGCTTTTCTGTGACTGGTGAGTACTCAACCAAGTCATTCTGAGAATAGTGTAT
GCGGCGACCGAGTTGCTCTTGCCCGGCGTCAATACGGGATAATACCGCGCCACATAGCAGAACT
TTAAAAGTGCTCATCATTGGAAAACGTTCTTCGGGGCGAAAACTCTCAAGGATCTTACCGCTGT
TGAGATCCAGTTCGATGTAACCCACTCGTGCACCCAACTGATCTTCAGCATCTTTTACTTTCAC
CAGCGTTTCTGGGTGAGCAAAAACAGGAAGGCAAAATGCCGCAAAAAAGGGAATAAGGGCGACA
CGGAAATGTTGAATACTCATACTCTTCCTTTTTCAATATTATTGAAGCATTTATCAGGGTTATT
GTCTCATGAGCGGATACATATTTGAATGTATTTAGAAAAATAAACAAATAGGGGTTCCGCGCAC
ATTTCCCCGAAAAGTGCCACCTGACGTCTAAGAAACCATTATTATCATGACATTAACCTATAAA
AATAGGCGTATCACGAGGCCCTTTCGTCTCGCGCGTTTCGGTGATGACGGTGAAAACCTCTGAC
ACATGCAGCTCCCGGAGACGGTCACAGCTTGTCTGTAAGCGGATGCCGGGAGCAGACAAGCCCG
TCAGGGCGCGTCAGCGGGTGTTGGCGGGTGTCGGGGCTGGCTTAACTATGCGGCATCAGAGCAG
ATTGTACTGAGAGTGCACCATAAAATTGTAAACGTTAATATTTTGTTAAAATTCGCGTTAAATT
TTTGTTAAATCAGCTCATTTTTTAACCAATAGACCGAAATCGGCAAAATCCCTTATAAATCAAA
AGAATAGCCCGAGATAGAGTTGAGTGTTGTTCCAGTTTGGAACAAGAGTCCACTATTAAAGAAC
GTGGACTCCAACGTCAAAGGGCGAAAAACCGTCTATCAGGGCGATGGCCCACTACGTGAACCAT
CACCCAAATCAAGTTTTTTGGGGTCGAGGTGCCGTAAAGCACTAAATCGGAACCCTAAAGGGAG
CCCCCGATTTAGAGCTTGACGGGGAAAGCCGGCGAACGTGGCGAGAAAGGAAGGGAAGAAAGCG
AAAGGAGCGGGCGCTAAGGCGCTGGCAAGTGTAGCGGTCACGCTGCGCGTAACCACCACACCCG
CCGCGCTTAATGCGCCGCTACAGGGCGCGTACTATGGTTGCTTTGACGTATGCGGTGTGAAATA
CCGCACAGATGCGTAAGGAGAAAATACCGCATCAGGCGCC
>human CD45 homology arm_long_5′-
SEQ ID NO: 6
TTTGGCTGGTCTAAAGTGACCTACACTGCCAGTAGGCTGGCATGTGGCTGCCATTTAGCTATGG
CAACAACAGTGAGTGGGCCACATGTCCCTCCTCATCCAGAAGACTAGCCCAGGCCTATTCACAT
TAAAGCAGCAAGTTCCACAAGGGAAAGAAGACTTGTGTGAGACCACTTGAGGCCCAGGCTTAAA
AGTGACACACATGTCTTCTTCTGTATGTTATTAGCCAAATAAATAAGTCATAAAGCCTGCCCAG
ATTCAAGGGGTAGGGAAATAGACTCCACTTCTTGAGAGGGCCTGCAAATTCACATTGCAAAGAA
ATGTGGATACAGGAAGGAAAATAAGTTTTATATTCTTGTAATCGATCTATCGTGTATACCCTCT
ATGTGGTAGTAACTGTAGATGGTCATCTGGGAATTAATCCTTATTCACAGTGTAAACTTAATTA
CTCACTAAAATATATAAAGCTTTTAATCATGTATGATATTGAGATTTCATATCTTGGTACTTAA
AAATGTATCAAATGCTTGCTATGTGCTCTTGCTATAAAGAGCTAATTGGTATGAGGGAAAGCCA
GGTATTTACTAATCAATGTAGTGAGTAAAATGACAGAAAAATTATAAGAAGAACATGAATGAGG
GCATTTAATTTAAACTTTAGGAATCAAGAAACGCTTCTCGAAGCAGTGATTCCTGCCCTGATTC
TTAAATAATGTGTAGGCATTAGACAGGAGGATAAGTACAAAACGTGGCATCATGAGCAAAGGCA
TGGAAATGGCCCATGAGCGGAGTGAACACTGGTTTGGGGTTGCTCCAAGGTAAAGTTCAAAAAG
TATCCTGCAGTCAACCCTTTAGCACCATAAAGAAACTAAATTATTTAGATGTTTTTATGAGAAC
ATATCAAAAAGTACTTTTCTGTCATCCAATACTTCCACAAATAAATCATTAGTTCTTGCTAATC
TTCATCTGGCATAAAAATAATGACATCAACTTTCTTCATGTAATTTCCCACTTAATTCCTTTAC
TAGGAGCAATATCAATTCCTATATGACGTCATTGCCAGCACCTACCCTGCTCAGAATGGACAAG
TAAAGAAAAACAACCATCAAGAAGATAAAATTGAATTTGATAATGAAGTGGACAAAGTAAAGCA
GGATGCTAATTGTGTTAATCCACTTGGTGCCCCAGAAAAGCTCCCTGAAGCAAAGGAACAGGCT
GAAGGTTCTGAACCCACGAGTGGCACTGAGGGGCCAGAACATTCTGTCAATGGTCCTGCAAGTC
CAGCTTTAAATCAAGGTTCA
>human CD45 homology arm_long_3′-
SEQ ID NO: 7
TAGGAAAAGACATAAATGAGGAAACTCCAAACCTCCTGTTAGCTGTTATTTCTATTTTTGTAGA
AGTAGGAAGTGAAAATAGGTATACAGTGGATTAATTAAATGCAGCGAACCAATATTTGTAGAAG
GGTTATATTTTACTACTGTGGAAAAATATTTAAGATAGTTTTGCCAGAACAGTTTGTACAGACG
TATGCTTATTTTAAAATTTTATCTCTTATTCAGTAAAAAACAACTTCTTTGTAATCGTTATGTG
TGTATATGTATGTGTGTATGGGTGTGTGTTTGTGTGAGAGACAGAGAAAGAGAGAGAATTCTTT
CAAGTGAATCTAAAAGCTTTTGCTTTTCCTTTGTTTTTATGAAGAAAAAATACATTTTATATTA
GAAGTGTTAACTTAGCTTGAAGGATCTGTTTTTAAAAATCATAAACTGTGTGCAGACTCAATAA
AATCATGTACATTTCTGAAATGACCTCAAGATGTCCTCCTTGTTCTACTCATATATATCTATCT
TATATAGTTTACTATTTTACTTCTAGAGATAGTACATAAAGGTGGTATGTGTGTGTATGCTACT
ACAAAAAAGTTGTTAACTAAATTAACATTGGGAAATCTTATATTCCATATATTAGCATTTAGTC
CAATGTCTTTTTAAGCTTATTTAATTAAAAAATTTCCAGTGAGCTTATCATGCTGTCTTTACAT
GGGGTTTTCAATTTTGCATGCTCGATTATTCCCTGTACAATATTTAAAATTTATTGCTTGATAC
TTTTGACAACAAATTAGGTTTTGTACAATTGAACTTAAATAAATGTCATTAAAATAAATAAATG
CAATATGTATTAATATTCATTGTATAAAAATAGAAGAATACAAACATATTTGTTAAATATTTAC
ATATGAAATTTAATATAGCTATTTTTATGGAATTTTTCATTGATATGAAAAATATGATATTGCA
TATGCATAGTTCCCATGTTAAATCCCATTCATAACTTTCATTAAAGCATTTACTTTGAATTTCT
CCAATGCTTAGAATGTTTTTACCAGGAATGGATGTCGCTAATCATAATAAAATTCAACCATTAT
TTTTTTCTTGTTTATAATACATTGTGTTATATGTTCAAATATGAAATGTGTATGCACCTATTGA
AATATGTTTAATGCATTTATTAACATTTGCAGGACACTTTTACAGGCC
>hCD45-long HA-T2A-GFP vector-
SEQ ID NO: 8
CCTGCAGGCAGCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCCATC
ACTAGGGGTTCCTGCGGCCAGAACCGGTTCATCTAGATTTGGCTGGTCTAAAGTGACCTACACT
GCCAGTAGGCTGGCATGTGGCTGCCATTTAGCTATGGCAACAACAGTGAGTGGGCCACATGTCC
CTCCTCATCCAGAAGACTAGCCCAGGCCTATTCACATTAAAGCAGCAAGTTCCACAAGGGAAAG
AAGACTTGTGTGAGACCACTTGAGGCCCAGGCTTAAAAGTGACACACATGTCTTCTTCTGTATG
TTATTAGCCAAATAAATAAGTCATAAAGCCTGCCCAGATTCAAGGGGTAGGGAAATAGACTCCA
CTTCTTGAGAGGGCCTGCAAATTCACATTGCAAAGAAATGTGGATACAGGAAGGAAAATAAGTT
TTATATTCTTGTAATCGATCTATCGTGTATACCCTCTATGTGGTAGTAACTGTAGATGGTCATC
TGGGAATTAATCCTTATTCACAGTGTAAACTTAATTACTCACTAAAATATATAAAGCTTTTAAT
CATGTATGATATTGAGATTTCATATCTTGGTACTTAAAAATGTATCAAATGCTTGCTATGTGCT
CTTGCTATAAAGAGCTAATTGGTATGAGGGAAAGCCAGGTATTTACTAATCAATGTAGTGAGTA
AAATGACAGAAAAATTATAAGAAGAACATGAATGAGGGCATTTAATTTAAACTTTAGGAATCAA
GAAACGCTTCTCGAAGCAGTGATTCCTGCCCTGATTCTTAAATAATGTGTAGGCATTAGACAGG
AGGATAAGTACAAAACGTGGCATCATGAGCAAAGGCATGGAAATGGCCCATGAGCGGAGTGAAC
ACTGGTTTGGGGTTGCTCCAAGGTAAAGTTCAAAAAGTATCCTGCAGTCAACCCTTTAGCACCA
TAAAGAAACTAAATTATTTAGATGTTTTTATGAGAACATATCAAAAAGTACTTTTCTGTCATCC
AATACTTCCACAAATAAATCATTAGTTCTTGCTAATCTTCATCTGGCATAAAAATAATGACATC
AACTTTCTTCATGTAATTTCCCACTTAATTCCTTTACTAGGAGCAATATCAATTCCTATATGAC
GTCATTGCCAGCACCTACCCTGCTCAGAATGGACAAGTAAAGAAAAACAACCATCAAGAAGATA
AAATTGAATTTGATAATGAAGTGGACAAAGTAAAGCAGGATGCTAATTGTGTTAATCCACTTGG
TGCCCCAGAAAAGCTCCCTGAAGCAAAGGAACAGGCTGAAGGTTCTGAACCCACGAGTGGCACT
GAGGGGCCAGAACATTCTGTCAATGGTCCTGCAAGTCCAGCTTTAAATCAAGGTTCAGAAGGTC
GTGGATCACTACTTACGTGCGGTGATGTAGAAGAGAATCCGGGTCCGGTCGACATGCCCGCCAT
GAAGATCGAGTGCCGCATCACCGGCACCCTGAACGGCGTGGAGTTCGAGCTGGTGGGCGGCGGA
GAGGGCACCCCCGAGCAGGGCCGCATGACCAACAAGATGAAGAGCACCAAAGGCGCCCTGACCT
TCAGCCCCTACCTGCTGAGCCACGTGATGGGCTACGGCTTCTACCACTTCGGCACCTACCCCAG
CGGCTACGAGAACCCCTTCCTGCACGCCATCAACAACGGCGGCTACACCAACACCCGCATCGAG
AAGTACGAGGACGGCGGCGTGCTGCACGTGAGCTTCAGCTACCGCTACGAGGCCGGCCGCGTGA
TCGGCGACTTCAAGGTGGTGGGCACCGGCTTCCCCGAGGACAGCGTGATCTTCACCGACAAGAT
CATCCGCAGCAACGCCACCGTGGAGCACCTGCACCCCATGGGCGATAACGTGCTGGTGGGCAGC
TTCGCCCGCACCTTCAGCCTGCGCGACGGCGGCTACTACAGCTTCGTGGTGGACAGCCACATGC
ACTTCAAGAGCGCCATCCACCCCAGCATCCTGCAGAACGGGGGCCCCATGTTCGCCTTCCGCCG
CGTGGAGGAGCTGCACAGCAACACCGAGCTGGGCATCGTGGAGTACCAGCACGCCTTCAAGACC
CCCATCGCCTTCGCCAGATCTCGAGCTCGATGAGTTTGGACAAACCACAACTAGAATGCAGTGA
AAAAAATGCTTTATTTGTGAAATTTGTGATGCTATTGCTTTATTTGTAACCATTATAAGCTGCA
ATAAACAAGTTACGCGTTAGGAAAAGACATAAATGAGGAAACTCCAAACCTCCTGTTAGCTGTT
ATTTCTATTTTTGTAGAAGTAGGAAGTGAAAATAGGTATACAGTGGATTAATTAAATGCAGCGA
ACCAATATTTGTAGAAGGGTTATATTTTACTACTGTGGAAAAATATTTAAGATAGTTTTGCCAG
AACAGTTTGTACAGACGTATGCTTATTTTAAAATTTTATCTCTTATTCAGTAAAAAACAACTTC
TTTGTAATCGTTATGTGTGTATATGTATGTGTGTATGGGTGTGTGTTTGTGTGAGAGACAGAGA
AAGAGAGAGAATTCTTTCAAGTGAATCTAAAAGCTTTTGCTTTTCCTTTGTTTTTATGAAGAAA
AAATACATTTTATATTAGAAGTGTTAACTTAGCTTGAAGGATCTGTTTTTAAAAATCATAAACT
GTGTGCAGACTCAATAAAATCATGTACATTTCTGAAATGACCTCAAGATGTCCTCCTTGTTCTA
CTCATATATATCTATCTTATATAGTTTACTATTTTACTTCTAGAGATAGTACATAAAGGTGGTA
TGTGTGTGTATGCTACTACAAAAAAGTTGTTAACTAAATTAACATTGGGAAATCTTATATTCCA
TATATTAGCATTTAGTCCAATGTCTTTTTAAGCTTATTTAATTAAAAAATTTCCAGTGAGCTTA
TCATGCTGTCTTTACATGGGGTTTTCAATTTTGCATGCTCGATTATTCCCTGTACAATATTTAA
AATTTATTGCTTGATACTTTTGACAACAAATTAGGTTTTGTACAATTGAACTTAAATAAATGTC
ATTAAAATAAATAAATGCAATATGTATTAATATTCATTGTATAAAAATAGAAGAATACAAACAT
ATTTGTTAAATATTTACATATGAAATTTAATATAGCTATTTTTATGGAATTTTTCATTGATATG
AAAAATATGATATTGCATATGCATAGTTCCCATGTTAAATCCCATTCATAACTTTCATTAAAGC
ATTTACTTTGAATTTCTCCAATGCTTAGAATGTTTTTACCAGGAATGGATGTCGCTAATCATAA
TAAAATTCAACCATTATTTTTTTCTTGTTTATAATACATTGTGTTATATGTTCAAATATGAAAT
GTGTATGCACCTATTGAAATATGTTTAATGCATTTATTAACATTTGCAGGACACTTTTACAGGC
CATCGATGCAGCGGCCGCTGCAGGCCGCAGGAACCCCTAGTGATGGAGTTGGCCACTCCCTCTC
TGCGCGCTCGCTCGCTCACTGAGGCCGGGCGACCAAAGGTCGCCCGACGCCCGGGCGGCCTCAG
TGAGCGAGCGAGCGCGCAGCTGCCTGCAGGACATGTGAGCAAAAGGCCAGCAAAAGGCCAGGAA
CCGTAAAAAGGCCGCGTTGCTGGCGTTTTTCCATAGGCTCCGCCCCCCTGACGAGCATCACAAA
AATCGACGCTCAAGTCAGAGGTGGCGAAACCCGACAGGACTATAAAGATACCAGGCGTTTCCCC
CTGGAAGCTCCCTCGTGCGCTCTCCTGTTCCGACCCTGCCGCTTACCGGATACCTGTCCGCCTT
TCTCCCTTCGGGAAGCGTGGCGCTTTCTCATAGCTCACGCTGTAGGTATCTCAGTTCGGTGTAG
GTCGTTCGCTCCAAGCTGGGCTGTGTGCACGAACCCCCCGTTCAGCCCGACCGCTGCGCCTTAT
CCGGTAACTATCGTCTTGAGTCCAACCCGGTAAGACACGACTTATCGCCACTGGCAGCAGCCAC
TGGTAACAGGATTAGCAGAGCGAGGTATGTAGGCGGTGCTACAGAGTTCTTGAAGTGGTGGCCT
AACTACGGCTACACTAGAAGAACAGTATTTGGTATCTGCGCTCTGCTGAAGCCAGTTACCTTCG
GAAAAAGAGTTGGTAGCTCTTGATCCGGCAAACAAACCACCGCTGGTAGCGGTGGTTTTTTTGT
TTGCAAGCAGCAGATTACGCGCAGAAAAAAAGGATCTCAAGAAGATCCTTTGATCTTTTCTACG
GGGTCTGACGCTCAGTGGAACGAAAACTCACGTTAAGGGATTTTGGTCATGAGATTATCAAAAA
GGATCTTCACCTAGATCCTTTTAAATTAAAAATGAAGTTTTAAATCAATCTAAAGTATATATGA
GTAAACTTGGTCTGACAGTTACCAATGCTTAATCAGTGAGGCACCTATCTCAGCGATCTGTCTA
TTTCGTTCATCCATAGTTGCCTGACTCCCCGTCGTGTAGATAACTACGATACGGGAGGGCTTAC
CATCTGGCCCCAGTGCTGCAATGATACCGCGAGACCCACGCTCACCGGCTCCAGATTTATCAGC
AATAAACCAGCCAGCCGGAAGGGCCGAGCGCAGAAGTGGTCCTGCAACTTTATCCGCCTCCATC
CAGTCTATTAATTGTTGCCGGGAAGCTAGAGTAAGTAGTTCGCCAGTTAATAGTTTGCGCAACG
TTGTTGCCATTGCTACAGGCATCGTGGTGTCACGCTCGTCGTTTGGTATGGCTTCATTCAGCTC
CGGTTCCCAACGATCAAGGCGAGTTACATGATCCCCCATGTTGTGCAAAAAAGCGGTTAGCTCC
TTCGGTCCTCCGATCGTTGTCAGAAGTAAGTTGGCCGCAGTGTTATCACTCATGGTTATGGCAG
CACTGCATAATTCTCTTACTGTCATGCCATCCGTAAGATGCTTTTCTGTGACTGGTGAGTACTC
AACCAAGTCATTCTGAGAATAGTGTATGCGGCGACCGAGTTGCTCTTGCCCGGCGTCAATACGG
GATAATACCGCGCCACATAGCAGAACTTTAAAAGTGCTCATCATTGGAAAACGTTCTTCGGGGC
GAAAACTCTCAAGGATCTTACCGCTGTTGAGATCCAGTTCGATGTAACCCACTCGTGCACCCAA
CTGATCTTCAGCATCTTTTACTTTCACCAGCGTTTCTGGGTGAGCAAAAACAGGAAGGCAAAAT
GCCGCAAAAAAGGGAATAAGGGCGACACGGAAATGTTGAATACTCATACTCTTCCTTTTTCAAT
ATTATTGAAGCATTTATCAGGGTTATTGTCTCATGAGCGGATACATATTTGAATGTATTTAGAA
AAATAAACAAATAGGGGTTCCGCGCACATTTCCCCGAAAAGTGCCACCTGACGTCTAAGAAACC
ATTATTATCATGACATTAACCTATAAAAATAGGCGTATCACGAGGCCCTTTCGTCTCGCGCGTT
TCGGTGATGACGGTGAAAACCTCTGACACATGCAGCTCCCGGAGACGGTCACAGCTTGTCTGTA
AGCGGATGCCGGGAGCAGACAAGCCCGTCAGGGCGCGTCAGCGGGTGTTGGCGGGTGTCGGGGC
TGGCTTAACTATGCGGCATCAGAGCAGATTGTACTGAGAGTGCACCATAAAATTGTAAACGTTA
ATATTTTGTTAAAATTCGCGTTAAATTTTTGTTAAATCAGCTCATTTTTTAACCAATAGACCGA
AATCGGCAAAATCCCTTATAAATCAAAAGAATAGCCCGAGATAGAGTTGAGTGTTGTTCCAGTT
TGGAACAAGAGTCCACTATTAAAGAACGTGGACTCCAACGTCAAAGGGCGAAAAACCGTCTATC
AGGGCGATGGCCCACTACGTGAACCATCACCCAAATCAAGTTTTTTGGGGTCGAGGTGCCGTAA
AGCACTAAATCGGAACCCTAAAGGGAGCCCCCGATTTAGAGCTTGACGGGGAAAGCCGGCGAAC
GTGGCGAGAAAGGAAGGGAAGAAAGCGAAAGGAGCGGGCGCTAAGGCGCTGGCAAGTGTAGCGG
TCACGCTGCGCGTAACCACCACACCCGCCGCGCTTAATGCGCCGCTACAGGGCGCGTACTATGG
TTGCTTTGACGTATGCGGTGTGAAATACCGCACAGATGCGTAAGGAGAAAATACCGCATCAGGC
GCC
>human CD45 homology arm (HA)_short_5′-
SEQ ID NO: 9
CAAAGGCATGGAAATGGCCCATGAGCGGAGTGAACACTGGTTTGGGGTTGCTCCAAGGTAAAGT
TCAAAAAGTATCCTGCAGTCAACCCTTTAGCACCATAAAGAAACTAAATTATTTAGATGTTTTT
ATGAGAACATATCAAAAAGTACTTTTCTGTCATCCAATACTTCCACAAATAAATCATTAGTTCT
TGCTAATCTTCATCTGGCATAAAAATAATGACATCAACTTTCTTCATGTAATTTCCCACTTAAT
TCCTTTACTAGGAGCAATATCAATTCCTATATGACGTCATTGCCAGCACCTACCCTGCTCAGAA
TGGACAAGTAAAGAAAAACAACCATCAAGAAGATAAAATTGAATTTGATAATGAAGTGGACAAA
GTAAAGCAGGATGCTAATTGTGTTAATCCACTTGGTGCCCCAGAAAAGCTCCCTGAAGCAAAGG
AACAGGCTGAAGGTTCTGAACCCACGAGTGGCACTGAGGGGCCAGAACATTCTGTCAATGGTCC
TGCAAGTCCAGCTTTAAATCAAGGTTCA
>human CD45 homology arm (HA)_short_3′-
SEQ ID NO: 10
TAGGAAAAGACATAAATGAGGAAACTCCAAACCTCCTGTTAGCTGTTATTTCTATTTTTGTAGA
AGTAGGAAGTGAAAATAGGTATACAGTGGATTAATTAAATGCAGCGAACCAATATTTGTAGAAG
GGTTATATTTTACTACTGTGGAAAAATATTTAAGATAGTTTTGCCAGAACAGTTTGTACAGACG
TATGCTTATTTTAAAATTTTATCTCTTATTCAGTAAAAAACAACTTCTTTGTAATCGTTATGTG
TGTATATGTATGTGTGTATGGGTGTGTGTTTGTGTGAGAGACAGAGAAAGAGAGAGAATTCTTT
CAAGTGAATCTAAAAGCTTTTGCTTTTCCTTTGTTTTTATGAAGAAAAAATACATTTTATATTA
GAAGTGTTAACTTAGCTTGAAGGATCTGTTTTTAAAAATCATAAACTGTGTGCAGACTCAATAA
AATCA
>hCD45-short HA-T2A-GFP vector-
SEQ ID NO: 11
CCTGCAGGCAGCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCCATC
ACTAGGGGTTCCTGCGGCCAGATCATCTAGACAAAGGCATGGAAATGGCCCATGAGCGGAGTGA
ACACTGGTTTGGGGTTGCTCCAAGGTAAAGTTCAAAAAGTATCCTGCAGTCAACCCTTTAGCAC
CATAAAGAAACTAAATTATTTAGATGTTTTTATGAGAACATATCAAAAAGTACTTTTCTGTCAT
CCAATACTTCCACAAATAAATCATTAGTTCTTGCTAATCTTCATCTGGCATAAAAATAATGACA
TCAACTTTCTTCATGTAATTTCCCACTTAATTCCTTTACTAGGAGCAATATCAATTCCTATATG
ACGTCATTGCCAGCACCTACCCTGCTCAGAATGGACAAGTAAAGAAAAACAACCATCAAGAAGA
TAAAATTGAATTTGATAATGAAGTGGACAAAGTAAAGCAGGATGCTAATTGTGTTAATCCACTT
GGTGCCCCAGAAAAGCTCCCTGAAGCAAAGGAACAGGCTGAAGGTTCTGAACCCACGAGTGGCA
CTGAGGGGCCAGAACATTCTGTCAATGGTCCTGCAAGTCCAGCTTTAAATCAAGGTTCAGAAGG
TCGTGGATCACTACTTACGTGCGGTGATGTAGAAGAGAATCCGGGTCCGGTCGACATGCCCGCC
ATGAAGATCGAGTGCCGCATCACCGGCACCCTGAACGGCGTGGAGTTCGAGCTGGTGGGCGGCG
GAGAGGGCACCCCCGAGCAGGGCCGCATGACCAACAAGATGAAGAGCACCAAAGGCGCCCTGAC
CTTCAGCCCCTACCTGCTGAGCCACGTGATGGGCTACGGCTTCTACCACTTCGGCACCTACCCC
AGCGGCTACGAGAACCCCTTCCTGCACGCCATCAACAACGGCGGCTACACCAACACCCGCATCG
AGAAGTACGAGGACGGCGGCGTGCTGCACGTGAGCTTCAGCTACCGCTACGAGGCCGGCCGCGT
GATCGGCGACTTCAAGGTGGTGGGCACCGGCTTCCCCGAGGACAGCGTGATCTTCACCGACAAG
ATCATCCGCAGCAACGCCACCGTGGAGCACCTGCACCCCATGGGCGATAACGTGCTGGTGGGCA
GCTTCGCCCGCACCTTCAGCCTGCGCGACGGCGGCTACTACAGCTTCGTGGTGGACAGCCACAT
GCACTTCAAGAGCGCCATCCACCCCAGCATCCTGCAGAACGGGGGCCCCATGTTCGCCTTCCGC
CGCGTGGAGGAGCTGCACAGCAACACCGAGCTGGGCATCGTGGAGTACCAGCACGCCTTCAAGA
CCCCCATCGCCTTCGCCAGATCTCGAGCTCGATGAGTTTGGACAAACCACAACTAGAATGCAGT
GAAAAAAATGCTTTATTTGTGAAATTTGTGATGCTATTGCTTTATTTGTAACCATTATAAGCTG
CAATAAACAAGTTACGCGTTAGGAAAAGACATAAATGAGGAAACTCCAAACCTCCTGTTAGCTG
TTATTTCTATTTTTGTAGAAGTAGGAAGTGAAAATAGGTATACAGTGGATTAATTAAATGCAGC
GAACCAATATTTGTAGAAGGGTTATATTTTACTACTGTGGAAAAATATTTAAGATAGTTTTGCC
AGAACAGTTTGTACAGACGTATGCTTATTTTAAAATTTTATCTCTTATTCAGTAAAAAACAACT
TCTTTGTAATCGTTATGTGTGTATATGTATGTGTGTATGGGTGTGTGTTTGTGTGAGAGACAGA
GAAAGAGAGAGAATTCTTTCAAGTGAATCTAAAAGCTTTTGCTTTTCCTTTGTTTTTATGAAGA
AAAAATACATTTTATATTAGAAGTGTTAACTTAGCTTGAAGGATCTGTTTTTAAAAATCATAAA
CTGTGTGCAGACTCAATAAAATCAATCGATGCATGCAGGCCGCAGGAACCCCTAGTGATGGAGT
TGGCCACTCCCTCTCTGCGCGCTCGCTCGCTCACTGAGGCCGGGCGACCAAAGGTCGCCCGACG
CCCGGGCGGCCTCAGTGAGCGAGCGAGCGCGCAGCTGCCTGCAGGACATGTGAGCAAAAGGCCA
GCAAAAGGCCAGGAACCGTAAAAAGGCCGCGTTGCTGGCGTTTTTCCATAGGCTCCGCCCCCCT
GACGAGCATCACAAAAATCGACGCTCAAGTCAGAGGTGGCGAAACCCGACAGGACTATAAAGAT
ACCAGGCGTTTCCCCCTGGAAGCTCCCTCGTGCGCTCTCCTGTTCCGACCCTGCCGCTTACCGG
ATACCTGTCCGCCTTTCTCCCTTCGGGAAGCGTGGCGCTTTCTCATAGCTCACGCTGTAGGTAT
CTCAGTTCGGTGTAGGTCGTTCGCTCCAAGCTGGGCTGTGTGCACGAACCCCCCGTTCAGCCCG
ACCGCTGCGCCTTATCCGGTAACTATCGTCTTGAGTCCAACCCGGTAAGACACGACTTATCGCC
ACTGGCAGCAGCCACTGGTAACAGGATTAGCAGAGCGAGGTATGTAGGCGGTGCTACAGAGTTC
TTGAAGTGGTGGCCTAACTACGGCTACACTAGAAGAACAGTATTTGGTATCTGCGCTCTGCTGA
AGCCAGTTACCTTCGGAAAAAGAGTTGGTAGCTCTTGATCCGGCAAACAAACCACCGCTGGTAG
CGGTGGTTTTTTTGTTTGCAAGCAGCAGATTACGCGCAGAAAAAAAGGATCTCAAGAAGATCCT
TTGATCTTTTCTACGGGGTCTGACGCTCAGTGGAACGAAAACTCACGTTAAGGGATTTTGGTCA
TGAGATTATCAAAAAGGATCTTCACCTAGATCCTTTTAAATTAAAAATGAAGTTTTAAATCAAT
CTAAAGTATATATGAGTAAACTTGGTCTGACAGTTACCAATGCTTAATCAGTGAGGCACCTATC
TCAGCGATCTGTCTATTTCGTTCATCCATAGTTGCCTGACTCCCCGTCGTGTAGATAACTACGA
TACGGGAGGGCTTACCATCTGGCCCCAGTGCTGCAATGATACCGCGAGACCCACGCTCACCGGC
TCCAGATTTATCAGCAATAAACCAGCCAGCCGGAAGGGCCGAGCGCAGAAGTGGTCCTGCAACT
TTATCCGCCTCCATCCAGTCTATTAATTGTTGCCGGGAAGCTAGAGTAAGTAGTTCGCCAGTTA
ATAGTTTGCGCAACGTTGTTGCCATTGCTACAGGCATCGTGGTGTCACGCTCGTCGTTTGGTAT
GGCTTCATTCAGCTCCGGTTCCCAACGATCAAGGCGAGTTACATGATCCCCCATGTTGTGCAAA
AAAGCGGTTAGCTCCTTCGGTCCTCCGATCGTTGTCAGAAGTAAGTTGGCCGCAGTGTTATCAC
TCATGGTTATGGCAGCACTGCATAATTCTCTTACTGTCATGCCATCCGTAAGATGCTTTTCTGT
GACTGGTGAGTACTCAACCAAGTCATTCTGAGAATAGTGTATGCGGCGACCGAGTTGCTCTTGC
CCGGCGTCAATACGGGATAATACCGCGCCACATAGCAGAACTTTAAAAGTGCTCATCATTGGAA
AACGTTCTTCGGGGCGAAAACTCTCAAGGATCTTACCGCTGTTGAGATCCAGTTCGATGTAACC
CACTCGTGCACCCAACTGATCTTCAGCATCTTTTACTTTCACCAGCGTTTCTGGGTGAGCAAAA
ACAGGAAGGCAAAATGCCGCAAAAAAGGGAATAAGGGCGACACGGAAATGTTGAATACTCATAC
TCTTCCTTTTTCAATATTATTGAAGCATTTATCAGGGTTATTGTCTCATGAGCGGATACATATT
TGAATGTATTTAGAAAAATAAACAAATAGGGGTTCCGCGCACATTTCCCCGAAAAGTGCCACCT
GACGTCTAAGAAACCATTATTATCATGACATTAACCTATAAAAATAGGCGTATCACGAGGCCCT
TTCGTCTCGCGCGTTTCGGTGATGACGGTGAAAACCTCTGACACATGCAGCTCCCGGAGACGGT
CACAGCTTGTCTGTAAGCGGATGCCGGGAGCAGACAAGCCCGTCAGGGCGCGTCAGCGGGTGTT
GGCGGGTGTCGGGGCTGGCTTAACTATGCGGCATCAGAGCAGATTGTACTGAGAGTGCACCATA
AAATTGTAAACGTTAATATTTTGTTAAAATTCGCGTTAAATTTTTGTTAAATCAGCTCATTTTT
TAACCAATAGACCGAAATCGGCAAAATCCCTTATAAATCAAAAGAATAGCCCGAGATAGAGTTG
AGTGTTGTTCCAGTTTGGAACAAGAGTCCACTATTAAAGAACGTGGACTCCAACGTCAAAGGGC
GAAAAACCGTCTATCAGGGCGATGGCCCACTACGTGAACCATCACCCAAATCAAGTTTTTTGGG
GTCGAGGTGCCGTAAAGCACTAAATCGGAACCCTAAAGGGAGCCCCCGATTTAGAGCTTGACGG
GGAAAGCCGGCGAACGTGGCGAGAAAGGAAGGGAAGAAAGCGAAAGGAGCGGGCGCTAAGGCGC
TGGCAAGTGTAGCGGTCACGCTGCGCGTAACCACCACACCCGCCGCGCTTAATGCGCCGCTACA
GGGCGCGTACTATGGTTGCTTTGACGTATGCGGTGTGAAATACCGCACAGATGCGTAAGGAGAA
AATACCGCATCAGGCGCC
>T2A peptide coding sequence-
SEQ ID NO: 12
GAAGGTCGTGGATCACTACTTACGTGCGGTGATGTAGAAGAGAATCCGGGTCCG
>AAVS1 sequence-
SEQ ID NO: 13
CACTTCAGGACAGCATGTTTGCTGCCTCCAGGGATCCTGTGTCCCCGAGCTGGGACCACCTTAT
ATTCCCAGGGCCGGTTAATGTGGCTCTGGTTCTGGGTACTTTTATCTGTCCCCTCCACCCCACA
GTGGGGCCACTAGGGACAGGATTGGTGACAGAAAAGCCCCATCCTTAGGCCTCCTCCTTCCTAG
TCTCCTGATATTGGGTCTAACCCCCACCTCCTGTTAGGCAGATTCCTTATCTGGTGACACACCC
CCATTTCCTGGA
>AAVS1 gRNA target sequence-
SEQ ID NO: 14
GGGGCCACTAGGGACAGGATTGG
>human CD45 gRNA-1 target sequence-
SEQ ID NO: 15
GCAAGTCCAGCTTTAAATCA
>human CD45 gRNA-2 target sequence-
SEQ ID NO: 16
ATAACAGCTAACAGGAGGTT
>human CD45 gRNA-3 target sequence-
SEQ ID NO: 17
CATAGGAAAAGACATAAATG
>human CD45 gRNA-4 target sequence-
SEQ ID NO: 18
AGCTTTAAATCAAGGTTCAT
>human CD45 gRNA-5 target sequence-
SEQ ID NO: 19
TATGAACCTTGATTTAAAGC
>human CD45 gRNA-6 target sequence-
SEQ ID NO: 20
TAGAAATAACAGCTAACAGG

1. An isolated nucleic acid comprising an expression construct comprising transgene encoding a gene product flanked by a 5′ homology arm and a 3′ homology arm, wherein the expression construct is flanked by adeno-associated virus (AAV) inverted terminal repeats (ITRs).

2. (canceled)

3. The isolated nucleic acid of claim 1, wherein the gene product comprises a therapeutic protein or a reporter protein.

4. The isolated nucleic acid of claim 3, wherein the therapeutic protein is useful for treating a hemoglobinopathy, optionally wherein the hemoglobinopathy is sickle cell disease.

5. (canceled)

6. The isolated nucleic acid of claim 3, wherein the therapeutic protein is a Hemoglobin Subunit Beta.

7. (canceled)

8. The isolated nucleic acid of claim 7, wherein the homology arms are specific for an AAVIS genomic safe harbor (GSH) site.

9. (canceled)

10. The isolated nucleic acid of claim 8, wherein the 5′ AAVS1 homology arm comprises the nucleic acid sequence set forth in SEQ ID NO: 1.

11. The isolated nucleic acid of claim 8, wherein the 3′ AAVS1 homology arm comprises the nucleic acid sequence set forth in SEQ ID NO: 2.

12-16. (canceled)

17. A recombinant adeno-associated virus (rAAV) comprising:
(i) the isolated nucleic acid of claim 1; and
(ii) an AAV capsid protein.

18. The rAAV of claim 17, wherein the AAV capsid protein is of a serotype selected from AAV1, AAV2, AAV3, AAV4, AAV5, AAV6, AAV7, AAV8, AAV9, or a variant thereof.

19-20. (canceled)

21. A pharmaceutical composition comprising the isolated nucleic acid of claim 1 and one or more guide RNAs (gRNAs).

22. The pharmaceutical composition of claim 21, wherein the one or more gRNAs comprise a region of complementarity the genomic locus that the homology arms are specific for.

23. The pharmaceutical composition of claim 21, wherein the one or more gRNAs specifically bind to an AAVIS genomic safe harbor (GSH) locus.

24. (canceled)

25. The pharmaceutical composition of claim 23, wherein the gRNAs specifically bind to the target sequence of the AAVIS locus as set forth in SEQ ID NO: 14.

26-28. (canceled)

29. A method for in vivo homology directed repair (HDR), the method comprising administering the isolated nucleic acid of claim 1, to a subject.

30. A method for in vitro homology directed repair (HDR), the method comprising administering the isolated nucleic acid of claim 1 to an ex vivo cell; and, optionally, introducing the cell into a subject.

31-42. (canceled)

43. The isolated nucleic acid of claim 1, wherein the isolated nucleic acid further comprises a nucleic acid sequence encoding a 2A peptide, wherein the nucleic acid sequence encoding the 2A peptide is located between the 5′ homology arm and the transgene.

44-45. (canceled)

46. The isolated nucleic acid of claim 1, wherein the 5′ and 3′ homology arms are specific for a genomic locus of CD45.

47. The isolated nucleic acid of claim 46, wherein the CD45 is human CD45.

48. The isolated nucleic acid of claim 47, wherein the 5′ homology arm specific for CD45 comprises the nucleic acid sequence as set forth in SEQ ID NO: 6 or SEQ ID NO: 9 and/or wherein the 3′ homology arm specific for CD45 comprises the nucleic acid sequence as set forth in SEQ ID NO: 7 or SEO ID NO: 10.

49-78. (canceled)
